(19) United States
(12) Patent Application Publication
(10) Pub. No.: US 2019/0161534 A1
(43) Pub. Date: May 30, 2019

(54) ANTI-GALACTAN II MONOCLONAL ANTIBODIES TARGETING KLEBSIELLA PNEUMONIAE

(71) Applicants: ARSANIS Biosciences GmbH, Vienna (AT); Max-Planck-Gesellschaft zur Förderung der Wissenschaften e. V., Munich (DE)

(72) Inventors: Valeria SZIJÁRTO, Vienna (AT); Gábor NAGY, Sopron (HU); Luis GUACHALLA, Vienna (AT); Eszter NAGY, Wien (AT); Tim ROLLENSKE, Heidelberg (DE); Hedda WARDEMANN, Heidelberg (DE)

(73) Assignees: ARSANIS Biosciences GmbH, Vienna (AT); Max-Planck-Gesellschaft zur Förderung der Wissenschaften e. V., Munich (DE)

(21) Appl. No.: 16/321,528

(22) PCT Filed: Aug. 11, 2017
(86) PCT No.: PCT/EP2017/070483

§ 371 Date: Jan. 29, 2019

(30) Foreign Application Priority Data
Aug. 12, 2016 (EP) 16183953.5

(51) Int. Cl. C07K 16/12; A61P 31/04; C12N 15/85

(52) U.S. Cl. CPC C07K 16/1228; A61P 31/04; A61K 38/00; C12N 2015/8518; C12N 15/85

(57) ABSTRACT

A cross-neutralizing monoclonal antibody that specifically recognizes a cross-reactive epitope of the lipopolysaccharide (LPS) antigen structure of Klebsiella pneumoniae, which is an O3b epitope, cross-reacting with an O3a epitope and an O3 epitope, wherein the antibody is characterized by specific CDR sequences or VH and VL sequences.

### FIELD OF THE INVENTION

The invention relates to a high affinity antibody targeting galactan-II (D-galactan-II, D-gal II, gal-II) of Klebsiella pneumoniae serotype O1, that shows neutralization of endotoxic activity of liberated lipopolysaccharide (LPS).

### BACKGROUND OF THE INVENTION

Klebsiella pneumoniae is a nosocomial opportunistic pathogen responsible for urinary tract infections, pneumonia, and septicaemia, which cause significant morbidity and mortality. The susceptible patients often have impaired immune functions unable to cope with invasive infections caused by this commensal enterobacterium.

Even more alarming is that multi-drug resistant (MDR) strains have recently emerged and spread globally, against which therapeutic options are limited. Monoclonal antibodies may represent a novel therapeutic approach. Nevertheless, molecular targets accessible on the surface of K. pneumonia are very limited given the bulky capsular polysaccharide that shields most surface antigens. On the other hand the readily accessible capsular polysaccharide shows high structural and hence antigenical variability that renders it non-attractive for broad spectrum antibacterial approaches.

The other major non-proteinaceous surface antigen is LPS that shows less variability than the capsular antigen. In K. pneumoniae there are less than 10 O-serogroups distinguished based on the structure of the LPS O-side chains. The most common serotype is O1, which was reported to be expressed by more than one third of all K. pneumoniae isolates (1;3). In the K. pneumoniae serotype O1, D-galactan II provides the epitope that defines the O1 antigen, and is characterized by the D-gal II repeat unit structure: [-3)-α-D-Galp-(1-3)-β-D-Galp-(1-].

The detailed biochemical structure of K. pneumoniae O1 antigen was described earlier by two independent groups (4;5). Polyclonal and murine monoclonal IgG2a antibodies against the K. pneumoniae O1 antigen were raised and characterized by the Trautmann group as described above (1-3). Based on their experimental data the therapeutic use of anti-O1 mAbs was suggested. It was shown that such antibodies induce opsonophagocytotic killing (OPK) (1) and afford protection in murine models of Klebsiella infections (2). Based on the opsonizing potential, the use of mAbs against galactan-II was implied as putative antibacterial strategy (2,3). Yet, Lepper et al. (FEMS Immunology and Medical Microbiology 2003, 35: 93-98) describe that naturally occurring O-antigen-specific antibodies, which are directed mainly against LPS side chain epitopes, do not appear to contribute significantly to phagocytic killing.

Given that the patient population susceptible for Klebsiella infections is typically immunocompromised this mode of action may be compromised in those who would benefit the most from passive immunization. Therefore, other modes of action such as direct complement mediated bactericidal activity or neutralization of endotoxic activity of LPS molecules would be desirable, though have not yet been described.

There is prior art for the detailed structural analysis of the O1 antigen as well as monoclonal antibodies against this structure. Affinity of binding was not determined, and prior art antibodies were selected for its opsonisation function, i.e. efficacy would rely on phagocyte function of the infected host (phagocytic killing). K. pneumonia, as an opportunistic pathogen, however, tends to infect immunocompromised and immunodeficient individuals whose phagocytic activity may be severely compromised.

Immunocompromised and immunodeficient patients are unable to develop a normal immune response. Immunodeficiencies can be primary (when genetic defects affect immune cell development or function) or secondary (when factors affect a host with an intrinsically normal immune system resulting in acquired immunodeficiency) and they can result from disorders of antibodies, lymphocytes, phagocytes, the complement system or combination of these factors.

As K. pneumoniae typically causes outbreaks in nosocomial settings, patients present at the same clinical ward, sharing medical equipment or personnel with a K. pneumoniae infected patient are at high risk of contracting infection.

As MDR resistant, even pan-resistant strains of K. pneumoniae are emerging, there is a high medical need for novel drugs against this pathogen. Moreover, mortality rates of invasive K. pneumoniae infections is high, even in case of appropriate antibiotic therapy, there may be a need for alternative therapeutic options, including antibodies recognizing targets of Klebsiella pneumoniae aiming at neutralizing endotoxin activity.

### SUMMARY OF THE INVENTION

It is the objective of the present invention to provide for an antibody directed against K. pneumoniae with improved relevance to target the pathogen, to be used for the prevention or therapy of K. pneumoniae infections.

The object is solved by the subject of the present invention.

According to the invention, there is provided a monoclonal antibody (mAb) that specifically recognizes Klebsiella pneumoniae serotype O1, which is capable of neutralizing the LPS endotoxin activity, wherein the antibody comprises any one of

a) the CDR1-CDR6 sequences of any of the antibodies listed in Table 1a or Table 1 b; or

b) the VH and VL sequences of any of the antibodies depicted in FIG. 2b; or

c) is a functionally active variant of a parent antibody that is characterized by the sequences of a) or b),

wherein the functionally active variant has a specificity to bind the same epitope as the parent antibody or to compete with the parent antibody, and comprises at least one functionally active CDR variant of any of the CDR1-CDR6 of the parent antibody (the parent CDR sequence), which functionally active CDR variant comprises at least one point mutation in the parent CDR sequence, and consists of the amino acid sequence that has at least 60% sequence identity with the parent CDR sequence, preferably at least 70%, at least 80%, or at least 90% sequence identity.

In particular, the antibody of the invention is a monoclonal antibody that specifically recognizes Klebsiella pneumoniae serotype O1, which is capable of neutralizing the LPS endotoxin activity, which antibody is selected from any of

a) an antibody comprising the CDR1-CDR6 sequences of any one of the antibodies listed in Table 1a or Table 1 b; or

b) an antibody comprising the the VH and VL sequences of any one of the antibodies depicted in FIG. 2b; or

c) an antibody which is a functionally active variant of a parent antibody that is any one of the antibodies of a) or b), which functionally active variant specifically recognizes Klebsiella pneumoniae serotype O1 and is capable of neutralizing the LPS endotoxin activity, and comprises at least one point mutation in any of the CDR, wherein the number of point mutations is either 0, 1, 2, or 3 point mutations in each of the CDR sequences, which has at least 60% sequence identity to the respective parent CDR sequence.

Specifically, the functionally active variant is provided wherein the sequence identity in each of the CDR sequences is at least 60% compared to the respective CDR sequences of the parent antibody.

In particular, the antibody described herein specifically recognizes the LPS side chain of K. pneumoniae serotype O1.

In particular, the antibody described herein specifically recognizes the D-galactan-II antigen within the LPS side chain of K. pneumoniae serotype O1 (the “01 antigen”), more specifically the gal-II epitope. Such antibody may as well recognize the same epitope or antigen which is expressed by (bacterial) cells other than K. pneumoniae.

Specifically, the antibody is an antibody characterized by the antigen binding site of any of the antibodies listed in FIG. 1, in particular an antibody which is

A)

selected from the group consisting of group members i) to ii), wherein

i)

is an antibody which comprises
a) CDR1 consisting of the amino acid sequence SEQ ID 9;
b) CDR2 consisting of the amino acid sequence SEQ ID 10;
c) CDR3 consisting of the amino acid sequence of SEQ ID 11;
d) CDR4 consisting of the amino acid sequence SEQ ID 15;
e) CDR5 consisting of the amino acid sequence SEQ ID 16; and
f) CDR6 consisting of the amino acid sequence of SEQ ID 17;

ii)

is an antibody which comprises
a) CDR1 consisting of the amino acid sequence SEQ ID 12;
b) CDR2 consisting of the amino acid sequence SEQ ID 13;
c) CDR3 consisting of the amino acid sequence of SEQ ID 14;
d) CDR4 consisting of the amino acid sequence SEQ ID 18;
e) CDR5 consisting of the amino acid sequence SEQ ID 19; and
f) CDR6 consisting of the amino acid sequence of SEQ ID 20;

wherein CDR sequences are designated according to the numbering system of Kabat;

or

B) an antibody which is the functionally active variant of a parent antibody that is any of the group members of A. In particular, the functionally active variant is characterized by the features further described herein.

Specifically, the CDR sequences according to Kabat as referred to herein are understood as those amino acid sequences of an antibody as determined according to Kabat nomenclature (see Kabat et al., Sequences of Proteins of Immunological Interest, 5^{th} Ed. Public Health Service, U.S. Department of Health and Human Services. (1991)).

Specifically, the antibody is

A)

selected from the group consisting of group members i) to ii), wherein

i)

is an antibody which comprises
a) CDR1 consisting of the amino acid sequence SEQ ID 21;
b) CDR2 consisting of the amino acid sequence SEQ ID 22;
c) CDR3 consisting of the amino acid sequence of SEQ ID 23;
d) CDR4 consisting of the amino acid sequence SEQ ID 27;
e) CDR5 consisting of the amino acid sequence SEQ ID 28; and
f) CDR6 consisting of the amino acid sequence of SEQ ID 29;

and

ii)

is an antibody which comprises
a) CDR1 consisting of the amino acid sequence SEQ ID 24;
b) CDR2 consisting of the amino acid sequence SEQ ID 25;
c) CDR3 consisting of the amino acid sequence of SEQ ID 26;
d) CDR4 consisting of the amino acid sequence SEQ ID 30;
e) CDR5 consisting of the amino acid sequence SEQ ID 31; and
f) CDR6 consisting of the amino acid sequence of SEQ ID 32;

wherein CDR sequences are designated according to the numbering system of IMGT;

or

B) an antibody which is the functionally active variant of a parent antibody that is any of the group members of A. In particular, the functionally active variant is characterized by the features further described herein.

Specifically, the CDR sequences according to IMGT as referred to herein are understood as those amino acid sequences of an antibody as determined according to the IMGT system (The international ImMunoGeneTics, Lefranc et al., 1999, Nucleic Acids Res. 27: 209-212).

According to a specific embodiment, the antibody is

A)

selected from the group consisting of group members i) to ii), wherein

i)

is an antibody which comprises
a) VH consisting of the amino acid sequence SEQ ID 5; and
b) VL consisting of the amino acid sequence SEQ ID 7;

and

ii)

is an antibody which comprises
a) VH consisting of the amino acid sequence SEQ ID 6; and
b) VL consisting of the amino acid sequence SEQ ID 8;

or

B) an antibody which is the functionally active variant of a parent antibody that is any of the group members of A. In particular, the functionally active variant is characterized by the features further described herein.

Specifically, the functionally active variants of the antibodies described herein is a functional variant of such antibody e.g., a functional variant which substantially has the same binding specificity as the exemplified antibodies listed in the tables of FIG. 1, comprising the binding site formed by the six CDR sequences provided therein, and/or formed by pairing respective VH and VL antibody domains, e.g. the VH and VL domains characterized by the sequences provided in FIG. 2.

For the purpose of providing variants, such antibodies are herein referred to as parent antibodies, and CDR or framework (FR) sequences are herein referred to as parent CDR or parent framework sequences. It is well understood that any antibody sequence as described herein is considered a “parent” sequence which can be subject to variation e.g., by one or more point mutations.

According to a specific aspect, the functional variant antibody binds the same epitope as the parent antibody.

According to a further specific aspect, the functional variant antibody comprises the same binding site as the parent antibody.

Specifically the antibody is a high affinity antibody binding the epitope with a K_{D} of less than 10⁻⁸M, preferably less than 5×10⁻⁹M, as determined by biolayer interferometry for bivalent binding (e.g. for a full-length IgG antibody), in particular using the method described in the Example 7, employing a fortéBIO Octet Red instrument and fortéBIO analysis. Such method specifically determines the avidity of binding, herein also referred to as “avid binding affinity”.

The high affinity of binding can be confirmed when determining the affinity for the respective Fab fragment (monovalent binding).

The high binding affinity as described herein specifically relates to any of avid binding affinity (as determined for a bivalent binder) and/or the affinity (as determined for the monovalent binder).

Typically, such antibodies as described herein and variant antibodies are high affinity antibodies binding the O1 antigen (in particular the O1 antigen bearing the D-galactan-II antigen) with a K_{D} of less than 10⁻⁹M (e.g., when determining avid binding affinity upon bivalent binding), and/or a K_{D} of less than 10⁻⁶M (e.g., when determining affinity upon monovalent binding), and competitively binding to the D-galactan-II epitope. Competition of binding is preferably determined by competition ELISA analysis or by biolayer interferometry (BLI) analysis.

Specifically, the avid binding affinity targeting the O1 polysaccharide antigen (incorporating the D-galactan-II epitope) is measured by BLI using a fortéBIO Octet Red instrument (ForteBio analysis) (e.g., Pall Life Sciences), such as exemplified herein. According to a specific embodiment, the antibody has an avid binding affinity to the O1 antigen with a K_{D} of less than 10⁻⁹M, or less than 5×10⁻⁹M.

Affinity matured variants of a parent antibody described herein (e.g., using the exemplified mAbs as parent mAbs) may be produced employing standard mutagenesis techniques, and may produce mAbs that are characterized by even higher (avid) binding affinities e.g., with a K_{D} less than 10⁻⁹M, or preferably less than 10⁻¹⁰ M, or preferably less than 10⁻¹¹ M e.g., with an affinity in the picomolar range. Variants of parent antibodies which are produced by affinity maturation, herein referred to as affinity-matured variants, may have an increased binding affinity, with a K_{D} difference of at least 1 log, or 2 logs, or 3 logs, as compared to the parent antibody. Affinity matured variants typically have an affinity to bind the antigen with a K_{D} of less than 10⁻⁹ M.

Specificity of binding the antigen is e.g., determined by an immune assay (ELISA, immunoblot, flow cytometry, BLI) using native O1 antigen or bacteria expressing the antigen as well as additional control antigen(s), to which the antibody does not significantly bind.

The antibody as described herein is specifically further characterized that it does not cross-react with any other K. pneumoniae antigen, and/or the antibody binds to any other K. pneumoniae antigen with a lower affinity, e.g. where the K_{D} difference to preferentially bind the O1 antigen over other K. pneumoniae antigens (other than the O1 antigen) is at least 2 logs, preferably at least 3 logs.

The antibody or the functional variant of any of the exemplified antibodies (parent antibodies) which competitively binds to any of the parent antibodies is specifically characterized by a relative inhibition of binding to its target, e.g., as determined by competition ELISA analysis or by biolayer interferometry (BLI), which relative inhibition is preferably greater than 30%.

Specifically, the exemplified antibodies and functional variants thereof are characterized by the high affinity of specifically binding the O1 antigen and in particular the D-galactan-II epitope and structure, resulting in a neutralizing activity, and advantageously also in bactericidal killing.

Specifically, the antibody is neutralizing endotoxin of Klebsiella pneumoniae strains expressing the the D-galactan-II epitope. It surprisingly turned out that such high affinity antibodies exhibited a potency of neutralizing the endotoxic activity of LPS molecules liberated from K. pneumoniae O1. Furthermore, direct (phagocyte-independent) bactericidal activity, i.e. not dependent on cellular immune status of the host was determined. Based on these novel modes of action, the antibody as described herein is particular suitable for medical use in treating an immunocompromised or immunodeficient patient population as add-on or standalone therapeutic in case of invasive infections by K. pneumoniae O1.

The antibodies are particularly suitable for developing a new preventive measure for individuals being at risk of acquiring an immunocompromised condition with decreased phagocytic function (e.g., cancer patients before chemotherapy or radiation therapy, patients undergoing immunosuppressive therapy, or patients with chronic infections, e.g. HIV patients) or at patients on clinical wards affected by K. pneumoniae outbreaks.

Specifically, the antibody neutralizes the endotoxic effect of bacteria expressing the corresponding specific LPS molecules in vivo. Its function may be determined by in vitro assays. The antibody is specifically effective against Klebsiella pneumoniae of the 01 type by neutralizing endotoxin functions e.g., as determined by an in vitro LAL assay, or toll-like receptor 4 (TLR4) reporter assay e.g., with at least 20% reduction in endotoxin activities in comparison to control samples (no antibody or irrelevant control mAb added).

The antibody may specifically neutralize lethal endotoxemia. Such functional activity may be determined in an appropriate in vivo model (e.g., the GaIN model of endotoxemia, such as described in reference 6). According to a certain aspect, the antibody neutralizes the targeted pathogen in animals, including both, human and non-human animals, and inhibits pathogenesis in vivo, preferably any models of primary and secondary bacteremia, pneumonia, urinary tract infection, liver abscess, peritonitis, or meningitis.

Specifically, the neutralization potency is at least the potency of any of the exemplified antibodies characterized by the CDR sequences identified in FIG. 1 and/or characterized by the VH and VL sequences identified in FIG. 2, which are used as reference antibody when determining the neutralization potency of functional variants.

Specifically, the antibody is characterized by a bactericidal complement dependent cytotoxicity (CDC) activity. Specifically, the antibody comprises the structure of an IgG1 or IgG3 antibody, preferably comprising the Fc of human IgG1 or IgG3. Specifically, the antibody is an IgG1 or IgG3 antibody.

As used herein, the CDC of an antibody is the reaction wherein one or more complement protein components recognize bound antibody on a target cell and subsequently cause lysis of the target cell.

Specifically, the antibody is characterized by the CDC activity to complement-mediated direct killing of the antigen-expressing bacterium in the circulation, as determined in serum, e.g. by a standard CDC assay, such as an in vitro serum bactericidal assay (SBA), e.g. with at least 20% killing of bacteria above the control samples (no antibody or irrelevant control mAb added).

It was surprising that an antibody as described herein was capable of LPS neutralization and directly killing the bacteria by CDC activity despite a natural resistance of K. pneumoniae serotype O1 to serum killing. The bactericidal activity is particularly relevant when treating patients with a phagocytic defect, or immunocompromised patients.

Besides, the antibody may specifically be effective against Klebsiella pneumoniae of the gal-II O-type by antibody mediated phagocytosis, e.g. as determined by an in vitro opsonophagocytotic killing assay (OPK), e.g. with at least 20% uptake of input bacteria or 20% lower end CFU count above the control samples (no antibody or irrelevant control mAb added).

Functionally active variant antibodies may differ in any of the VH or VL sequences, or share the common VH and VL sequences, and comprise modifications in the respective FR. The variant antibody derived from the parent antibody by mutagenesis may be produced by methods well-known in the art.

Functional variants of an antibody may specifically be engineered to obtain CDR mutated antibodies (including at least one CDR variant) e.g., to improve the affinity of an antibody. Specifically, the functionally active variant is a functionally active CDR variant which comprises at least one point mutation in the parent CDR sequence, and comprises or consists of the amino acid sequence that has at least 60% sequence identity with the parent CDR sequence, preferably at least 70%, at least 80%, at least 90% sequence identity.

A specific variant is e.g., a human or artificial variant of the parent antibody, wherein the parent CDR sequences are incorporated into human or artificial framework sequences (e.g. of non-human origin, such as human framework sequences including one or more point mutations), wherein optionally 1, 2, 3, or 4 amino acid residues of each of the parent CDR sequences may be further mutated by introducing point mutations to improve the stability, specificity and affinity of the parent or humanized antibody.

According to a specific aspect, the antibody comprises artificial CDR and framework sequences e.g., of non-human origin, wherein at least one of the CDR and framework sequences includes one or more point mutations such as to obtain artificial, non-naturally occurring sequences.

According to a certain aspect, the antibody is any one of a full-length antibody, an antibody fragment thereof, or a fusion protein, each comprising at least VH and VL antibody domains incorporating a binding site recognizing the D-galactan-II epitope. Specifically, the antibody is any of full-length IgG1, a bispecific IgG1, or a F(ab′)2-fragment.

Specifically, the antibody is a human antibody or a derivative thereof incorporating artificial or animal sequences (other than human), e.g. a human IgG antibody, or an antibody comprising human CDR sequences or any functional CDR variant thereof and an animal (non-human) framework, such as to obtain an animalized, e.g. caninized antibody.

Specifically, the antibody described herein is a fully human antibody.

The antibodies described in the examples are of human origin, or an affinity matured variant thereof, specifically wherein the antibody is a non-naturally occurring antibody which comprises an artificial amino acid sequence. Variants comprising artificial sequences (non-naturally occurrsing) may be obtained by mutagenesis or as further described herein.

Specifically, the antibody is any one of an IgA, IgM, or an IgG isotype switch variant thereof, e.g. IgA to IgG isotype switch variant, or IgM to IgG isotype switch variant. Fc portions can be of any immunoglobulin isotype, and in particular of an IgG (e.g., an IgG1) antibody

According to a specific aspect, the antibody of the invention comprises CDR and framework sequences, wherein the framework sequences include human, artificial or animal sequences. Specifically the antibody comprises one or more constant domains, which are of an IgG antibody e.g., of an IgG1, IgG2, IgG3, or IgG4 subtype, or of an IgA1, IgA2, IgD, IgE, or IgM antibody.

It is feasible that variant VH or VL domains e.g., with modifications in the respective FR or CDR sequences as compared to the VH and VL, respectively, of any of the antibodies as shown in FIG. 2b (herein refererd to as “parent” VH or “parent VL”) may be used, which are functionally active e.g., binding to the same epitope or comprising the same binding site or having the same binding characteristics as the parent antibody. It is also feasible that some of the FR or CDR sequences of the antibodies described herein may be exchanged by those of other antibodies.

Specifically, the variant VH or variant VL may be provided, which comprises

a) the set of 6 CDR (CDR1-6) sequences of the parent VH or VL, or the set of 6 CDR (CDR1-6) sequences, wherein at least one of the CDR sequences is a functionally active CDR variant of the parent CDR as further described herein; and

b) FR sequences characterized by at least 60% sequence identity with the FR sequences of the parent VH or VL, preferably at least 70%, at least 80%, or at least 90% sequence identity.

Specifically the antibody comprises a functionally active CDR variant of any of the CDR sequences listed in FIG. 1, wherein the functionally active CDR variant comprises at least one of

a) 1, 2, or 3 point mutations in the parent CDR sequence; and/or

b) 1 or 2 point mutations in any of the four C-terminal or four N-terminal, or four centric amino acid positions of the parent CDR sequence; and/or

c) at least 60% sequence identity with the parent CDR sequence, preferably at least 60% sequence identity in each of the CDR1-CDR6 sequences;

preferably wherein the functionally active CDR variant comprises 1 or 2 point mutations in any CDR sequence consisting of less than 4 or 5 amino acids.

Specifically, the functionally active variant differs from the parent antibody in at least one point mutation in the amino acid sequence, preferably in the CDR, wherein the number of point mutations in each of the CDR amino acid sequences is either 0, 1, 2 or 3.

According to a specific aspect, the point mutation is any of an amino acid substitution, deletion and/or insertion of one or more amino acids.

According to a specific aspect, the antibody is provided for use in treating a subject at risk of or suffering from K. pneumoniae infection or colonization to limit the infection in the subject or to ameliorate a disease condition resulting from said infection, preferably for treatment or prophylaxis of any of primary and secondary bacteremia, pneumonia, urinary tract infection, liver abscess, peritonitis, or meningitis.

According to a further specific aspect, the antibody is bactericidal in vitro and/or in vivo, and is specifically killing the targeted pathogen in animals, including both, human and non-human animals, and inhibits pathogenesis in vivo, preferably any models of primary and secondary bacteremia, pneumonia, urinary tract infection, liver abscess, peritonitis, or meningitis.

Therefore, the invention further provides for a method of treating a subject by administering an effective amount of the antibody in the respective indications.

Specifically, the subject is a human being. Specifically, the subject is any human being who is healthy or suffering from a disease. Specifically, the human being is an immunodeficient, in particular an immunocompromised or immunosuppressed patient, or a contact thereof.

Specifically, the subject is of a host group characterized by an impaired phagocyte number and/or function, which host group is any of

a) patients suffering from inherited or acquired primary or secondary immunodeficiency;

b) patients selected from the group consisting of neonates younger than 28 days of age, elderly patients older than 65 years of age, patients suffering from Diabetes mellitus, renal failure, cirrhosis, Trisomie 21, trauma, or HIV, or patients who have undergone surgical interventions or systemic treatment with corticosteroids; or

c) patients admitted to hospital or hospital personnel, in particular at an acute-care or intensive care unit, with a risk of contracting infection upon exposure to a patient suffering from K. pneumoniae disease.

Specifically, the antibody is used to prevent nosocomial or iatrogenic outbreaks of K. pneumoniae disease.

According to a specific embodiment, the antibody is administered at a prophylactically effective dose to prevent bacteremia, preferably less than 1 mg/kg.

According to another specific embodiment, the antibody is administered in a therapeutically effective dose to treat bacteremia, preferably less than 10 mg/kg.

Specifically, the antibody is provided for use according to the invention, wherein a systemic infection or colonization with Klebsiella pneumoniae of the gal-II O-type in a subject is determined ex vivo by contacting a biological sample of said subject with the antibody, wherein a specific immune reaction of the antibody determines the infection or colonization.

Specifically, the subject is suffering from endotoxemia caused by Klebsiella pneumoniae. According to a specific aspect, immunotherapy using the antibody of the invention may effectively protect against live bacterial challenge, e.g. as determined in various animal models. Antibodies described herein were particularly used for passive immunization eliciting protection in a mouse model of bacteremia.

The invention further provides for a pharmaceutical preparation comprising the antibody as described herein, and a pharmaceutically acceptable carrier or excipient in a parenteral (e.g., i.v. or i.m.) formulation.

The invention further provides for an isolated nucleic acid encoding the antibody as described herein.

The invention further provides for an expression cassette or a plasmid comprising a coding sequence to express a proteinaceous construct, such as comprising or consisting of a polypeptide or protein, or a protein derivative, comprising the binding site or the a VH and/or VL of the antibody as described herein. The invention further provides for a host cell comprising an expression cassette or a plasmid as described herein.

The invention further provides for a method of producing the antibody as described herein, wherein the host cell is cultivated or maintained under conditions to produce said antibody.

Specifically preferred is a host cell and a production method employing such host cell, which host cell comprises
the plasmid or expression cassette of the invention, which incorporates a coding sequence to express the antibody light chain; and
the plasmid or expression cassette of the invention, which incorporates a coding sequence to express the antibody heavy chain.

### FIGURES

FIG. 1: Tables 1a and 1 b:

Table 1a: CDR sequences of exemplified monoclonal antibodies (mAbs), wherein CDR sequences are designated according to the numbering system of Kabat;

Table 1b: CDR sequences of exemplified monoclonal antibodies (mAbs), wherein CDR sequences are designated according to the numbering system of IMGT;

The nomenclature as used herein shall have the following meaning:

VH CDR1=CDR1

VH CDR2=CDR2

VH CDR3=CDR3

VL CDR4=CDR4=VL CDR1

VL CDR5=CDR5=VL CDR2

VL CDR6=CDR6=VL CDR3

Antibody designated as KcPBO1-196 is herein also referred to as MPG-196.

FIG. 2: VH and VL sequences of exemplified mAbs: nucleotide sequences (FIG. 2a), and amino acid sequences (FIG. 2b).

FIG. 3: Identification of fully human anti-Klebsiella pneumoniae O1 O-antigen antibodies (a) Schematic linear chemical structure of the biotinylated O1 O-antigen bait; Box shows repetitive units; CP=common pare=(4-1)αHep or H. (b) Flow cytometric analysis and isolation of single 7-AAD⁻, CD19⁺, CD27⁺, O1 O-antigen-reactive human peripheral blood B cells (top) compared to a control staining using non-biotinylated O1 O-antigen. (c) Number of IGHV somatic hypermutations of memory B cells from peripheral blood of 8 individuals; enlarged open circle and open triangle indicate the number of somatic hypermutations for KcPBO1-196 and KePBO1-084, respectively. Concentration-dependent binding of KcPBO1-196 and KePBO1-084 to the biotinylated O-antigen bait by ELISA (d) and binding of KcPBO1-196 and KePBO1-084 to whole 01 LPS by Immunoblot (e); mG053=negative control. (f) Area under curve (AUC) from ELISA data, as shown in (d), to biotinylated O1 O-antigen and Streptavidin (SA); lines connect the same antibody; b,d-f: Representative data from at least two independent experiments; black solid lines show mean; error bars=standard deviation; dashed lines=negative cut-off OD.

FIG. 3: Ig gene features (table)

FIG. 4: Immunoblot with mAb MPG-196. One μg LPS purified from a panel of O1, O2, and O3 strains was separated and blotted onto PVDF membranes. Binding of MPG-196 (herein also referred to as KcPBO1-196) to the separated and blotted LPS samples was detected by an HRP conjugated anti-human IgG specific secondary antibody.

FIG. 5: Surface staining of K. pneumoniae O1 strains. A: Surface binding of mAb MPG-196 was tested on a panel of O1 strains expressing different capsular (K−) types by flow cytometry. Fluorescence intensity for the Alexa Fluor 488 anti-human mAb shown under FL1 and for the DNA dye Syto62 under FL4 represents the quantitative surface staining. B: surface staining of mAbs MPG-196 and 8E9 (a low affinity O1-specific mAb used for comparison reasons) were compared on three different O1:K2 strains. Histograms show fluorescence intensity measured.

FIG. 6: Protection by 01 specific mAbs in the mouse model of K. pneumoniae bacteremia. Mice were immunized prophylactically i.p. with either MPG-196 or 8E9 (O1-specific mAbs) or with an irrelevant control mAb. Survival was monitored following a subsequent intravenous lethal challenge (5×10⁶ CFU from a mid-log culture in LB (lysogeny broth)) with O1:K2 strain (ATCC 43816).

FIG. 7: Serum bactericidal assay. K. pneumoniae O1 strain PCM-37 (Polish collection of microbes) was cultured in 50% depleted human serum samples in the presence of various concentrations of specific or control mAbs. Bactericidal activity is expressed as percentage of recovered bacteria at the end vs the beginning (To) of the incubation period (3 h) as determined by plating aliquots at both timepoints.

FIG. 8: Endotoxin neutralization in vitro. A commercial cell line expressing the human TLR-4 receptor complex was used to report endotoxin signalling by purified LPS extracted from a K. pneumoniae O1 strain. Inhibition of signalling was tested with different concentrations of O1-specific mAb or polymyxin B (positive control) or an irrelevant control mAb (negative control).

FIG. 9: Protective efficacy of 01 mAbs in a murine endotoxemia model. Mice were prophylactically immunized with the indicated mAbs and doses. One day later mice were sensitized to endotoxin (GaIN, i.p., 20 mg/mouse) and challenged i.v. with 2×10³ CFU of O1:K2 strain 52145 grown to mid-log phase in LB. Lethality was monitored for 14 days.

### DETAILED DESCRIPTION

The term “antibody” as used herein shall refer to polypeptides or proteins that consist of or comprise antibody domains, which are understood as constant and/or variable domains of the heavy and/or light chains of immunoglobulins, with or without a linker sequence. Polypeptides are understood as antibody domains, if comprising a beta-barrel structure consisting of at least two beta-strands of an antibody domain structure connected by a loop sequence. Antibody domains may be of native structure or modified by mutagenesis or derivatization e.g., to modify the antigen binding properties or any other property, such as stability or functional properties, such as binding to the Fc receptors FcRn and/or Fc gamma receptor.

The antibody as used herein has a specific binding site to bind one or more antigens or one or more epitopes of such antigens, specifically comprising a CDR binding site of a single variable antibody domain, such as VH, VL or VHH, or a binding site of pairs of variable antibody domains, such as a VL/VH pair, an antibody comprising a VL/VH domain pair and constant antibody domains, such as Fab, F(ab′), (Fab)₂, scFv, Fv, or a full length antibody.

The term “antibody” as used herein shall particularly refer to antibody formats comprising or consisting of single variable antibody domain, such as VH, VL or VHH, or combinations of variable and/or constant antibody domains with or without a linking sequence or hinge region, including pairs of variable antibody domains, such as a VL/VH pair, an antibody comprising or consisting of a VL/VH domain pair and constant antibody domains, such as heavy-chain antibodies, Fab, F(ab′), (Fab)₂, scFv, Fd, Fv, or a full-length antibody e.g., of an IgG (e.g., an IgG1, IgG2, IgG3, or IgG4 subclass), IgA (e.g. an IgA1 or IgA2 subclass), IgD, IgE, or IgM isotype antibody. The term “full length antibody” can be used to refer to any antibody molecule comprising at least most of the Fc domain and other domains commonly found in a naturally occurring antibody monomer. This phrase is used herein to emphasize that a particular antibody molecule is not an antibody fragment.

The term “antibody” shall specifically include antibodies in the isolated form, which are substantially free of other antibodies directed against different target antigens or comprising a different structural arrangement of antibody domains. Still, an isolated antibody may be comprised in a combination preparation, containing a combination of the isolated antibody e.g., with at least one other antibody, such as monoclonal antibodies or antibody fragments having different specificities.

The term “antibody” shall apply to antibodies of animal origin, including human species, such as mammalian, including human, murine, rabbit, goat, lama, cow and horse, or avian, such as hen, which term shall particularly include recombinant antibodies which are based on a sequence of animal origin e.g., human sequences.

The term “antibody” further applies to chimeric antibodies with sequences of origin of different species, such as sequences of murine and human origin.

The term “chimeric” as used with respect to an antibody refers to those antibodies wherein one portion of each of the amino acid sequences of heavy and light chains is homologous to corresponding sequences in antibodies derived from a particular species, while the remaining segment of the chain is homologous to corresponding sequences in another species or class. Typically the variable region of both light and heavy chains mimics the variable regions of antibodies derived from one species of mammals, while the constant portions are homologous to sequences of antibodies derived from another. For example, the variable region can be derived from presently known sources using readily available B-cells or hybridomas from non-human host organisms in combination with constant regions derived from, for example, human cell preparations.

The term “antibody” may further apply to humanized antibodies.

The term “humanized” as used with respect to an antibody refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions in the variable domains. Antigen-binding sites may be wild-type or modified e.g., by one or more amino acid substitutions, preferably modified to resemble human immunoglobulins more closely. Some forms of humanized antibodies preserve all CDR sequences (for example a humanized mouse antibody which contains all six CDRs from the mouse antibody). Other forms have one or more CDRs which are altered with respect to the original antibody.

The term “antibody” further applies to human antibodies.

The term “human” as used with respect to an antibody, is understood to include antibodies having variable and constant regions derived from human germline immunoglobulin sequences. The human antibody of the invention may include amino acid residues not encoded by human germline immunoglobulin sequences (e.g., mutations introduced by random or site-specific mutagenesis in vitro or by somatic mutation in vivo), for example in the CDRs. Human antibodies include antibodies isolated from human immunoglobulin libraries or from animals transgenic for one or more human immunoglobulin genes or derived from human B cells by immunoglobulin gene cloning and recombinant antibody expression or from immortalized human B cell lines.

The term “fully human antibody” as used herein refers to a human antibody, which is composed of only human parts, in particular human CDR, human FR, and human constant regions, each originating from a human source, e.g. cells expressing human antibody sequences, libraries displaying human antibody sequences, or genes encoding human antibody sequences. Fully human antibodies may be naturally-occurring antibodies or artificial antibodies, which are understood as being composed of parts, each obtained from a different origin, thus, not occurring in nature. Exemplary artificial fully human antibodies are human switch variants of human antibodies, wherein at least one constant region is obtained from a human antibody of a different isotype.

The term “antibody” specifically applies to antibodies of any isotype or subclass. Depending on the amino acid sequence of the constant domain of their heavy chains, antibodies can be assigned to the major classes of antibodies IgA, IgD, IgE, IgG, and IgM, and several of these may be further divided into subclasses, e.g., IgG1, IgG2, IgG3, IgG4, IgA1, and IgA2.

The term further applies to monoclonal or polyclonal antibodies, specifically a recombinant antibody, which term includes all antibodies and antibody structures that are prepared, expressed, created or isolated by recombinant means, such as antibodies originating from animals e.g., mammalians including human, that comprises genes or sequences from different origin e.g., murine, chimeric, humanized antibodies, or hybridoma derived antibodies. Further examples refer to antibodies isolated from a host cell transformed to express the antibody, or antibodies isolated from a recombinant, combinatorial library of antibodies or antibody domains, or antibodies prepared, expressed, created or isolated by any other means that involve splicing or fusing antibody gene sequences to other DNA sequences.

It is understood that the term “antibody” also refers to derivatives of an antibody, in particular functionally active derivatives. An antibody derivative is understood as any combination of one or more antibody domains or antibodies and/or a fusion protein, in which any domain of the antibody may be fused at any position of one or more other proteins, such as other antibodies e.g., a binding structure comprising CDR loops, a receptor polypeptide, but also ligands, scaffold proteins, enzymes, toxins and the like. A derivative of the antibody may be obtained by association or binding to other substances by various chemical techniques such as covalent coupling, electrostatic interaction, di-sulphide bonding etc. The other substances bound to the antibody may be lipids, carbohydrates, nucleic acids, organic and inorganic molecules or any combination thereof (e.g., PEG, prodrugs or drugs). In a specific embodiment, the antibody is a derivative comprising an additional tag allowing specific interaction with a biologically acceptable compound. There is not a specific limitation with respect to the tag usable in the present invention, as far as it has no or tolerable negative impact on the binding of the antibody to its target. Examples of suitable tags include His-tag, Myc-tag, FLAG-tag, Strep-tag, Calmodulin-tag, GST-tag, MBP-tag, and S-tag. In another specific embodiment, the antibody is a derivative comprising a label. The term “label” as used herein refers to a detectable compound or composition which is conjugated directly or indirectly to the antibody so as to generate a “labeled” antibody. The label may be detectable by itself e.g., radioisotope labels or fluorescent labels, or, in the case of an enzymatic label, may catalyze chemical alteration of a substrate compound or composition which is detectable.

The preferred derivatives as described herein are functionally active with regard to antigen binding and endotoxin neutralization, preferably which have a potency to combat K. pneumoniae e.g., as determined in an SBA, OPK or LAL assay, or to protect against bacterial challenge or to neutralize endotoxemia.

The preferred derivatives as described herein are further functionally active to combat K. pneumoniae, e.g. as determined in a CDC (SBA), and/or OPK assay, or to protect against bacterial challenge.

Specifically, an antibody derived from an antibody of the invention may comprise at least one or more of the CDR regions or CDR variants thereof being functionally active in differentially binding to the O1 antigen, e.g. specifically or selectively binding the O1 antigen.

Antibodies derived from a parent antibody or antibody sequence, such as a parent CDR or FR sequence, are herein particularly understood as mutants or variants obtained by e.g., in silico or recombinant engineering or else by chemical derivatization or synthesis.

It is understood that the term “antibody” also refers to variants of an antibody, including antibodies with functionally active CDR variants of a parent CDR sequence, and functionally active variant antibodies of a parent antibody.

Specifically, an antibody derived from an antibody as described herein may comprise at least 3 CDRs of the heavy chain variable region and at least 3 CDRs of the light chain variable region, with at least one point mutation in at least one of the CDR or FR regions, or in the constant region of the HC or LC, being functionally active e.g., specifically binding the O1 antigen.

The term “variant” shall particularly refer to antibodies, such as mutant antibodies or fragments of antibodies e.g., obtained by mutagenesis methods, in particular to delete, exchange, introduce inserts into a specific antibody amino acid sequence or region or chemically derivatize an amino acid sequence e.g., in the constant domains to engineer the antibody stability, effector function or half-life, or in the variable domains to improve antigen-binding properties e.g., by affinity maturation techniques available in the art. Any of the known mutagenesis methods may be employed, including point mutations at desired positions e.g., obtained by randomization techniques. In some cases positions are chosen randomly e.g., with either any of the possible amino acids or a selection of preferred amino acids to randomize the antibody sequences. The term “mutagenesis” refers to any art recognized technique for altering a polynucleotide or polypeptide sequence. Preferred types of mutagenesis include error prone PCR mutagenesis, saturation mutagenesis, or other site directed mutagenesis.

The term “variant” shall specifically encompass functionally active variants.

The term “functionally active variant” of a CDR sequence as used herein, is understood as a “functionally active CDR variant”, and the “functionally active variant” of an antibody as used herein, is understood as “functionally active antibody variant”. The functionally active variant means a sequence resulting from modification of this sequence (a parent antibody or a parent sequence) by insertion, deletion or substitution of one or more amino acids, or chemical derivatization of one or more amino acid residues in the amino acid sequence, or nucleotides within the nucleotide sequence, or at either or both of the distal ends of the sequence e.g., in a CDR sequence the N-terminal and/or C-terminal 1, 2, 3, or 4 amino acids, and/or the centric 1, 2, 3, or 4 amino acids (i.e. in the midst of the CDR sequence), and which modification does not affect, in particular impair, the activity of this sequence. In the case of a binding site having specificity to a selected target antigen, the functionally active variant of an antibody would still have the predetermined binding specificity, though this could be changed e.g., to change the fine specificity to a specific epitope, the affinity, the avidity, the Kon or Koff rate, etc. For example, an affinity matured antibody is specifically understood as a functionally active variant antibody. Hence, the modified CDR sequence in an affinity matured antibody is understood as a functionally active CDR variant.

For example, a CDR variant includes an amino acid sequence modified by at least one amino acid in the CDR region, wherein said modification can be a chemical or a partial alteration of the amino acid sequence, which modification permits the variant to retain the biological characteristics of the unmodified sequence. A partial alteration of the CDR amino acid sequence may be by deletion or substitution of one to several amino acids e.g., 1, 2, 3, 4 or 5 amino acids, or by addition or insertion of one to several amino acids e.g., 1, 2, 3, 4 or 5 amino acids, or by a chemical derivatization of one to several amino acids e.g., 1, 2, 3, 4 or 5 amino acids, or combination thereof.

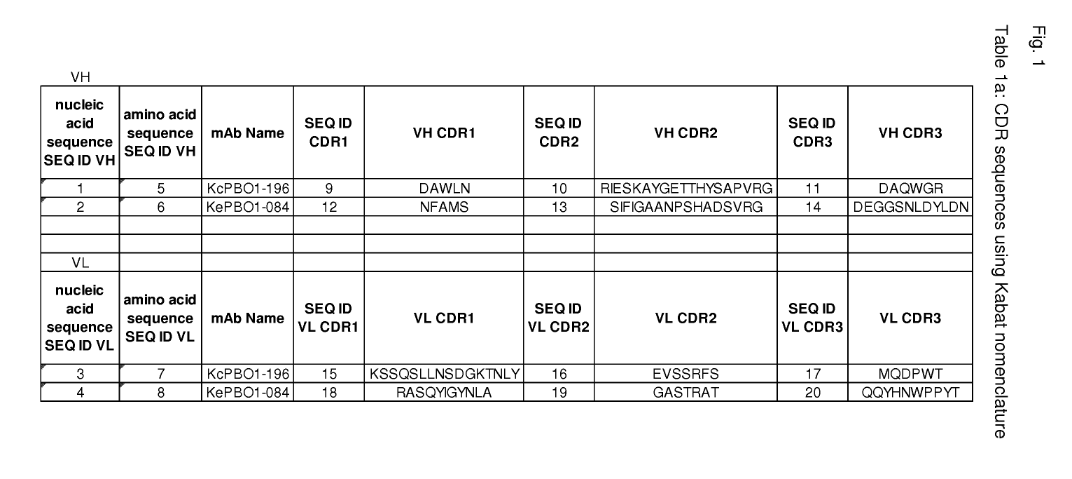

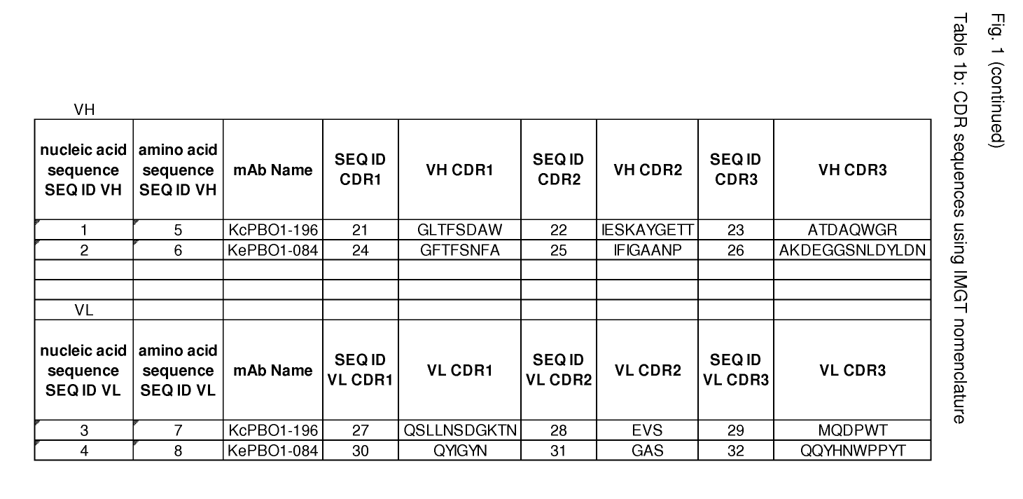

Functionally active variants may be obtained e.g., by changing the sequence of a parent antibody e.g., an antibody comprising the same binding site as any of the antibodies listed in FIG. 1, but with modifications within an antibody region besides the binding site, or derived from such parent antibody by a modification within the binding site but that does not impair the antigen binding, and preferably would have substantially the same biological activity as the parent antibody or even an improved activity, including the ability to specifically or selectively bind the O1 antigen.

In particular, the functionally active variants as described herein have the potency to specifically recognize K. pneumoniae O1, in particular recognizing the gal-II antigen of K. pneumoniae O1 and a neutralizing potency, such as an endotoxin neutralization function in a LAL assay e.g., with substantially the same biological activity, as determined by the specific binding assay or functional test to target K. pneumoniae.

Specifically, the functionally active variants as described herein have the potency to specifically bind gal-II antigen of K. pneumoniae O1, and the CDC activity to kill K. pneumoniae bacteria of the 01 serotype in the circulation/in serum.

Functionally active variants of exemplary (parent) antibodies may be obtained, e.g. by changing the sequence of a parent antibody, e.g. an antibody comprising the same binding site as any of the parent antibodies as described herein, but with modifications within an antibody region besides the binding site, or derived from such parent antibody by a modification within the binding site but that does not impair the antigen binding, and preferably would have substantially the same biological activity as the parent antibody or even an improved activity, including the ability to specifically or selectively bind O1 antigen of K. pneumoniae, and the neutralizing activity, and optionally the bactericidal CDC activity or potency of complement mediated killing in an SBA assay. Optionally, the functionally active variants may further include a potency of an antibody mediated phagocytosis in an OPK assay, e.g. with substantially the same biological activity, as determined by the specific binding assay or functional test to target (MDR) K. pneumoniae.

Antibodies combating or killing K. pneumoniae or neutralizing its endotoxins are able to limit or prevent infection and/or to ameliorate a disease condition resulting from such infection, or to inhibit K. pneumoniae pathogenesis, in particular dissemination and replication into or within sterile body compartments/sites of the host. In this regard, the neutralizing and bactericidal antibody as described herein, is also understood as being a “protective antibody” meaning that the antibody is responsible for immunity to an infectious agent observed in active or passive immunity. In particular, protective antibodies as described herein are possibly used for therapeutic purposes, e.g. for prophylaxis or therapy, to prevent, ameliorate, treat or at least partially arrest disease symptoms, side effects or progression of disease induced by a pathogen. Specifically, protective antibodies are able to kill or impede replication of live K. pneumoniae cells by e.g. inducing CDC or opsonophagocytic activities, or remove whole bacterial cells or the LPS molecules thereof from the sterile body sites following therapeutic applications (i.e. given on an established infection). Alternatively, prophylactically applied protective antibodies inhibit establishment of an infection (i.e. spread of K. pneumoniae from non-sterile sites to sterile body compartments) by one of the above mentioned or other mechanisms.

The term “substantially the same” with regard to binding a target antigen or biological activity as used herein refers to the activity as indicated by substantially the same activity being at least 20%, at least 50%, at least 75%, at least 90% e.g., at least 100%, or at least 125%, or at least 150%, or at least 175%, or e.g., up to 200%, or even a higher activity as determined for the comparable or parent antibody.

Specifically, the functionally active variants of an antibody of the invention have the potency to bind the O1 antigen with a high affinity, and the specificity to preferentially the galactan-II antigen relative to other (non-galactan-II antigens) of K. pneumoniae. Preferred variants are not binding to other antigens of K. pneumoniae, with a K_{D} value difference of at least 2 logs, preferably at least 3 logs, and further include endotoxin neutralization function in a LAL or TLR4 signaling assay, such as to achieve significant reduction of endotoxin activity relative to control samples not containing the antibody e.g., with substantially the same biological activity, as determined by the specific binding assay or functional test to target K. pneumoniae. The significant reduction of activity in functional in vitro assays typically means the reduction of at least 50%, preferably at least 60%, 70%, 80%, 90%, 95% or 98% up to complete reduction of about 100% (+/−1%).

In a preferred embodiment the functionally active variant of a parent antibody

a) is a biologically active fragment of the antibody, the fragment comprising at least 50% of the sequence of the molecule, preferably at least 60%, at least 70%, at least 80%, at least 90%, or at least 95% and most preferably at least 97%, 98% or 99%;

b) is derived from the antibody by at least one amino acid substitution, addition and/or deletion, wherein the functionally active variant has a sequence identity to the molecule or part of it, such as an antibody of at least 50% sequence identity, preferably at least 60%, more preferably at least 70%, more preferably at least 80%, still more preferably at least 90%, even more preferably at least 95% and most preferably at least 97%, 98% or 99%; and/or

c) consists of the antibody or a functionally active variant thereof and additionally at least one amino acid or nucleotide heterologous to the polypeptide or the nucleotide sequence.

In one preferred embodiment of the invention, the functionally active variant of the antibody as described herein is essentially identical to the variant described above, but differs from its polypeptide or the nucleotide sequence, respectively, in that it is derived from a homologous sequence of a different species. These are referred to as naturally occurring variants or analogs.

The term “functionally active variant” also includes naturally occurring allelic variants, as well as mutants or any other non-naturally occurring variants. As is known in the art, an allelic variant is an alternate form of a (poly) peptide that is characterized as having a substitution, deletion, or addition of one or more amino acids that does essentially not alter the biological function of the polypeptide.

Functionally active variants may be obtained by sequence alterations in the polypeptide or the nucleotide sequence e.g., by one or more point mutations, wherein the sequence alterations retains or improves a function of the unaltered polypeptide or the nucleotide sequence, when used in combination of the invention. Such sequence alterations can include, but are not limited to, (conservative) substitutions, additions, deletions, mutations and insertions.

Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

A point mutation is particularly understood as the engineering of a polynucleotide that results in the expression of an amino acid sequence that differs from the non-engineered amino acid sequence in the substitution or exchange, deletion or insertion of one or more single (non-consecutive) or doublets of amino acids for different amino acids.

Preferred point mutations refer to the exchange of amino acids of the same polarity and/or charge. In this regard, amino acids refer to twenty naturally occurring amino acids encoded by sixty-four triplet codons. These 20 amino acids can be split into those that have neutral charges, positive charges, and negative charges:

The “neutral” amino acids are shown below along with their respective three-letter and single-letter code and polarity:

Alanine: (Ala, A) nonpolar, neutral;

Asparagine: (Asn, N) polar, neutral;

Cysteine: (Cys, C) nonpolar, neutral;

Glutamine: (Gln, Q) polar, neutral;

Glycine: (Gly, G) nonpolar, neutral;

Isoleucine: (Ile, I) nonpolar, neutral;

Leucine: (Leu, L) nonpolar, neutral;

Methionine: (Met, M) nonpolar, neutral;

Phenylalanine: (Phe, F) nonpolar, neutral;

Proline: (Pro, P) nonpolar, neutral;

Serine: (Ser, S) polar, neutral;

Threonine: (Thr, T) polar, neutral;

Tryptophan: (Trp, W) nonpolar, neutral;

Tyrosine: (Tyr, Y) polar, neutral;

Valine: (Val, V) nonpolar, neutral; and

Histidine: (His, H) polar, positive (10%) neutral (90%).

The “positively” charged amino acids are:

Arginine: (Arg, R) polar, positive; and

Lysine: (Lys, K) polar, positive.

The “negatively” charged amino acids are:

Aspartic acid: (Asp, D) polar, negative; and

Glutamic acid: (Glu, E) polar, negative.

“Percent (%) amino acid sequence identity” with respect to the antibody sequences and homologs described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

The term “antigen-binding site” or “binding site” refers to the part of an antibody that participates in antigen binding. The antigen binding site is formed by amino acid residues of the N-terminal variable (“V”) regions of the heavy (“H”) and/or light (“L”) chains, or the variable domains thereof. Three highly divergent stretches within the V regions of the heavy and light chains, referred to as “hypervariable regions”, are inter-posed between more conserved flanking stretches known as framework regions, The antigen-binding site provides for a surface that is complementary to the three-dimensional surface of a bound epitope or antigen, and the hypervariable regions are referred to as “complementarity-determining regions”, or “CDRs.” The binding site incorporated in the CDRs is herein also called “CDR binding site”.

The term “antigen” as used herein interchangeably with the terms “target” or “target antigen” shall refer to a whole target molecule or a fragment of such molecule recognized by an antibody binding site. Specifically, substructures of an antigen e.g., a polypeptide or carbohydrate structure, generally referred to as “epitopes” e.g., B-cell epitopes or T-cell epitope, which are immunologically relevant, may be recognized by such binding site. Specific antigens incorporating the galactan-II epitope comprise carbohydrate (mannan) structures and may be provided as isolated antigens optionally provided on an artificial carrier, or else in the form of K. pneumoniae cells expressing the antigens or cell fractions thereof.

The term “epitope” as used herein shall in particular refer to a molecular structure which may completely make up a specific binding partner or be part of a specific binding partner to a binding site of an antibody. An epitope may either be composed of a carbohydrate, a peptidic structure, a fatty acid, an organic, biochemical or inorganic substance or derivatives thereof and any combinations thereof. If an epitope is comprised in a peptidic structure, such as a peptide, a polypeptide or a protein, it will usually include at least 3 amino acids, preferably 5 to 40 amino acids, and more preferably between about 10-20 amino acids. Epitopes can be either linear or conformational epitopes. A linear epitope is comprised of a single segment of a primary sequence of a polypeptide or carbohydrate chain. Linear epitopes can be contiguous or overlapping.

Conformational epitopes are comprised of amino acids or carbohydrates brought together by folding the polypeptide to form a tertiary structure and the amino acids are not necessarily adjacent to one another in the linear sequence. Specifically and with regard to polypeptide antigens a conformational or discontinuous epitope is characterized by the presence of two or more discrete amino acid residues, separated in the primary sequence, but assembling to a consistent structure on the surface of the molecule when the polypeptide folds into the native protein/antigen.

Herein the term “epitope” shall particularly refer to the single carbohydrate epitope of the O1 antigen recognized by an antibody as described herein.

The term “expression” is understood in the following way. Nucleic acid molecules containing a desired coding sequence of an expression product such as e.g., an antibody as described herein, and control sequences such as e.g., a promoter in operable linkage, may be used for expression purposes. Hosts transformed or transfected with these sequences are capable of producing the encoded proteins. In order to effect transformation, the expression system may be included in a vector; however, the relevant DNA may also be integrated into the host chromosome. Specifically the term refers to a host cell and compatible vector under suitable conditions e.g., for the expression of a protein coded for by foreign DNA carried by the vector and introduced to the host cell.

Coding DNA is a DNA sequence that encodes a particular amino acid sequence for a particular polypeptide or protein such as e.g., an antibody. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors will often include one or more replication systems for cloning or expression, one or more markers for selection in the host e.g., antibiotic resistance, and one or more expression cassettes.

“Vectors” used herein are defined as DNA sequences that are required for the transcription of cloned recombinant nucleotide sequences, i.e. of recombinant genes and the translation of their mRNA in a suitable host organism.

An “expression cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”.

Expression vectors comprise the expression cassette and additionally usually comprise an origin for autonomous replication in the host cells or a genome integration site, one or more selectable markers (e.g., an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The term “vector” as used herein includes autonomously replicating nucleotide sequences as well as genome integrating nucleotide sequences. A common type of vector is a “plasmid”, which generally is a self-contained molecule of double-stranded DNA that can readily accept additional (foreign) DNA and which can readily be introduced into a suitable host cell. A plasmid vector often contains coding DNA and promoter DNA and has one or more restriction sites suitable for inserting foreign DNA. Specifically, the term “vector” or “plasmid” refers to a vehicle by which a DNA or RNA sequence (e.g., a foreign gene) can be introduced into a host cell, so as to transform the host and promote expression (e.g., transcription and translation) of the introduced sequence.

The term “host cell” as used herein shall refer to primary subject cells transformed to produce a particular recombinant protein, such as an antibody as described herein, and any progeny thereof. It should be understood that not all progeny are exactly identical to the parental cell (due to deliberate or inadvertent mutations or differences in environment), however, such altered progeny are included in these terms, so long as the progeny retain the same functionality as that of the originally transformed cell. The term “host cell line” refers to a cell line of host cells as used for expressing a recombinant gene to produce recombinant polypeptides such as recombinant antibodies. The term “cell line” as used herein refers to an established clone of a particular cell type that has acquired the ability to proliferate over a prolonged period of time. Such host cell or host cell line may be maintained in cell culture and/or cultivated to produce a recombinant polypeptide.

The term “isolated” or “isolation” as used herein with respect to a nucleic acid, an antibody or other compound shall refer to such compound that has been sufficiently separated from the environment with which it would naturally be associated, so as to exist in “substantially pure” form. “Isolated” does not necessarily mean the exclusion of artificial or synthetic mixtures with other compounds or materials, or the presence of impurities that do not interfere with the fundamental activity, and that may be present, for example, due to incomplete purification. In particular, isolated nucleic acid molecules of the present invention are also meant to include those which are not naturally occurring e.g., codon-optimized nucleic acids or cDNA, or chemically synthesized.

Likewise, the isolated antibody of the invention is specifically non-naturally occurring e.g., as provided in a combination preparation with another antibody or active agent, which combination does not occur in nature, or an optimized or affinity-maturated variant of a naturally occurring antibody, or an antibody with a framework-region which is engineered to improve the manufacturability of the antibody. By such optimizing or engineering the antibody comprises one or more synthetic sequences or characteristics, which would not be found in the context of the antibody in nature.

With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

With reference to polypeptides or proteins, such as isolated antibodies or epitopes of the invention, the term “isolated” shall specifically refer to compounds that are free or substantially free of material with which they are naturally associated such as other compounds with which they are found in their natural environment, or the environment in which they are prepared (e g. cell culture) when such preparation is by recombinant DNA technology practiced in vitro or in vivo. Isolated compounds can be formulated with diluents or adjuvants and still for practical purposes be isolated—for example, the polypeptides or polynucleotides can be mixed with pharmaceutically acceptable carriers or excipients when used in diagnosis or therapy. In particular, the isolated antibody of the invention differs from polyclonal serum preparations raised against K. pneumoniae strains, because it is provided in the isolated and purified form, preferably provided in a preparation comprising the isolated antibody as the only active substance. This does not preclude, however, that the isolated antibody is provided in a combination product comprising a limited number of further well-defined (isolated) antibodies. Isolated antibodies may as well be provided on a solid, semi-liquid or liquid carrier, such as beads.

The term “neutralizing” or “neutralization” is used herein in the broadest sense and refers to any molecule that inhibits a pathogen, such as K. pneumoniae from infecting a subject, or to inhibit the pathogen from promoting infections by producing endotoxins, or to inhibit the endotoxins from exerting their biological activity, irrespective of the mechanism by which neutralization is achieved. Neutralization can be achieved, e.g., by an antibody that inhibits the colonization by K. pneumoniae of mucosal surfaces, invasion to sterile body sites, and eliciting adverse biological signals (in worst case inducing septic shock) in the host.

In the strict sense neutralization means, inhibiting the binding of specific LPS to its cognate receptor (e.g., Toll-like receptor-4 complex) and hence eliciting biological activity. This neutralization potency is typically determined in a standard assay e.g., an in vitro or in vivo neutralization assay e.g., a LAL test, or TLR-4 based assays, where the inhibition of endotoxin's biological activity is measured e.g., by colorimetry.

The term “O1 antigen”, also referred to as “galactan-II”, “gal-II” or “D-gal II” as used herein shall refer to the carbohydrate structure of the LPS O-antigen of K. pneumoniae comprising a galactose polymer and a structure comprising at least one of the repeat unit: [-3)-α-D-Galp-(1-3)-β-D-Galp-(1-]. Of note, the gal-II structure may also be expressed by organisms other than K. pneumoniae or respective cells, thus, can be a target of interest when combatting diseases mediated by such organisms or cells.

The respective O-antigen comprising the gal-II structure is herein referred to as “O1 antigen” which includes the “gal-II epitope” specifically being recognized by a O1 specific antibody as described herein. The O1 antigen is understood as the outer part of the LPS of K. pneumoniae of the O1-type (K. pneumoniae O1), which is the surface accessible antigenic carbohydrate structure comprising one or more specific gal-II epitopes incorporated therein.

“Specific” binding, recognizing or targeting as used herein, means that the binder e.g., antibody or antigen-binding portion thereof, exhibits appreciable affinity for the target antigen or a respective epitope in a heterogeneous population of molecules. Thus, under designated conditions (e.g., immunoassay), a binder specifically binds to the target the O1 antigen and does not bind in a significant amount to other molecules present in a sample. The specific binding means that binding is selective in terms of target identity, high, medium or low binding affinity or avidity, as selected. Selective binding is usually achieved if the binding constant or binding dynamics is at least 10-fold different (understood as at least 1 log difference), preferably the difference is at least 100-fold (understood as at least 2 logs difference), and more preferred a least 1000-fold (understood as at least 3 logs difference) as compared to another target.

Preferred antibodies as described herein are binding the O1 antigen, with a high affinity, in particular with a high on and/or a low off rate, or a high avidity of binding (avid binding affinity). The binding affinity of an antibody is usually characterized in terms of the concentration of the antibody, at which half of the antigen binding sites are occupied, known as the dissociation constant (Kd, or K_{D}). Usually a binder is considered a high affinity binder with a K_{D}<10⁻⁶ M or K_{D}<10⁻⁷ M as determined using a monovalent binder, or K_{D}<10⁻⁸ M as determined using a bivalent binder, in some cases e.g., for therapeutic purposes higher affinities e.g., with a K_{D}<10⁻⁸ M or even a K_{D}<10⁻⁹ M (as determined using a monovalent binder), or K_{D}<10⁻⁹ M, or even a K_{D}<10⁻¹⁰ M (as determined using a bivalent binder).

Yet, in a particularly preferred embodiment the individual antigen binding affinities are of medium affinity e.g., with a K_{D} of higher than 10⁻⁶, such as the avid binding affinity (as determined using a bivalent binder).

Medium affinity binders may be provided and affinity matured, if necessary.

Affinity maturation is the process by which antibodies with increased affinity for a target antigen are produced. Any one or more methods of preparing and/or using affinity maturation libraries available in the art may be employed in order to generate affinity matured antibodies in accordance with various embodiments of the invention disclosed herein. Exemplary such affinity maturation methods and uses, such as random mutagenesis, bacterial mutator strains passaging, site-directed mutagenesis, mutational hotspots targeting, parsimonious mutagenesis, antibody shuffling, light chain shuffling, heavy chain shuffling, CDR1 and/or CDR1 mutagenesis, and methods of producing and using affinity maturation libraries amenable to implementing methods and uses in accordance with various embodiments of the invention disclosed herein, include, for example, those disclosed in: Prassler et al. (2009); Immunotherapy, Vol. 1(4), pp. 571-583; Sheedy et al. (2007), Biotechnol. Adv., Vol. 25(4), pp. 333-352; WO2012/009568; WO2009/036379; WO2010/105256; US2002/0177170; WO2003/074679.

With structural changes of an antibody, including amino acid mutagenesis or as a consequence of somatic mutation in immunoglobulin gene segments, variants of a binding site to an antigen are produced and selected for greater affinities. Affinity matured antibodies may exhibit a several logfold greater affinity than a parent antibody. Single parent antibodies may be subject to affinity maturation. Alternatively pools of antibodies with similar binding affinity to the target antigen may be considered as parent structures that are varied to obtain affinity matured single antibodies or affinity matured pools of such antibodies.

The preferred affinity matured variant of an antibody as described herein exhibits at least a 2-fold increase in affinity of binding, preferably at least a 5, preferably at least 10, preferably at least 50, or preferably at least 100-fold increase. The affinity maturation may be employed in the course of the selection campaigns employing respective libraries of parent molecules, either with antibodies having medium binding affinity to obtain the antibody of the invention having the specific target binding property of a binding affinity K_{D}<10⁻⁹ M (e.g., avid binding affinity as determined using a bivalent binder). Alternatively, the affinity (e.g. avid binding affinity as determined using a bivalent binder) may be even more increased by affinity maturation of the antibody according to the invention to obtain the high values corresponding to a K_{D} of less than 10⁻⁹ M, preferably less than 10⁻¹⁰ M or even less than 10⁻¹¹ M, most preferred in the picomolar range.

In certain embodiments binding affinity is determined by an affinity ELISA assay. In certain embodiments binding affinity is determined by a BIAcore, BLI, ForteBio or MSD assays. In certain embodiments binding affinity is determined by a kinetic method. In certain embodiments binding affinity is determined by an equilibrium/solution method.

Use of the term “having the same specificity”, “having the same binding site” or “binding the same epitope” indicates that equivalent monoclonal antibodies exhibit the same or essentially the same, i.e. similar immunoreaction (binding) characteristics and compete for binding to a pre-selected target binding sequence. The relative specificity of an antibody molecule for a particular target can be relatively determined by competition assays e.g., as described in Harlow, et al., ANTIBODIES: A LABORATORY MANUAL, Cold Spring Harbor Laboratory Press, Cold Spring Harbor, N.Y., 1988).

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “compete” with each other for binding of their respective epitope(s). Antibodies that compete with any of the exemplified antibodies for binding the O1 antigen are particularly encompassed by the present invention.

“Competitively binding” or “competition” herein means a greater relative inhibition than about 30%, e.g., as determined by competition ELISA analysis or by ForteBio or BLI analysis. It may be desirable to set a higher threshold of relative inhibition as criteria of what is a suitable level of competition in a particular context e.g., where the competition analysis is used to select or screen for new antibodies designed with the intended function of the binding of the antigen. Thus, for example, it is possible to set criteria for the competitive binding, wherein at least 40% relative inhibition is detected, or at least 50%, at least 60%, at least 70%, at least 80%, at least 90% or even at least 100%, before an antibody is considered sufficiently competitive.

The term “K. pneumoniae infection” and “K. pneumoniae colonization” is understood in the following way: Klebsiella pneumoniae is a Gram-negative, bacterium that is a member of the family Enterobacteriaceae. It is a ubiquitous bacterium, which can also colonize the human host, typically in the intestines or the upper airways. Being an opportunistic pathogen, from these sites it can invade sterile body sites in case not properly controlled by the immune system. Uncontrolled bacterial replication at these sites will induce inflammation, in a great part, mediated by the endotoxin (i.e. LPS) molecules released from K. pneumoniae. In case of bacteremia, endotoxin molecules may trigger septic shock.

K. pneumoniae colonization means that the subject has a sufficiently high concentration of K. pneumoniae bacteria at a site that they can be detected, yet the bacteria are causing no signs or symptoms. Colonization can persist for a long period of time, with resolution influenced by the immune response to the organism, competition at the site from other organisms and, sometimes, use of antimicrobials.

In general, bacteremia caused by K. pneumoniae may be successfully treated with known conventional antibacterial therapy, such as treatment with antibiotics, steroid and non-steroid inhibitors of inflammation. The present invention provides for a new immunotherapy, employing antibodies specifically recognizing K. pneumoniae, which is optionally combined with anti-bacterial or anti-inflammatory therapy. Exemplary antibiotics used for treating patients with K. pneumoniae infection are aminoglycosides, cephalosporines, aminopenicilines, carbapenems, fluoroquinolons, tygecycline, colistin, etc.

Multi-drug resistant (MDR) K. pneumoniae is particularly understood as those strains demonstrating resistance to three or more classes of antibiotics e.g., the following agents/groups: penicillins, cephalosporins, carbapenems, aminoglycosides, tetracyclines, fluoroquinolones, nitrofurantoin, trimethoprim (and its combinations), fosfomycin, polymixins, chloramphenicol, azthreonam, or tigecycline.

With the recent emergence of antibiotic-resistant strains, treating bacteremia of this nature has become significantly more difficult. Patients who develop K. pneumoniae disease have longer hospital and ICU stays, high mortality, and greater health care costs than patients without K. pneumoniae disease. Patient care may be improved and nosocomial infections may be reduced by preventing, rather than treating, K. pneumoniae disease prophylaxis when a patient is heavily colonized by K. pneumoniae.

K. pneumoniae disease is specifically understood as a disease caused by K. pneumoniae infection. Such diseases include local and systemic disease. Severe cases of disease are e.g., primary and secondary bacteremia, pneumonia, urinary tract infection, liver abscess, peritonitis, or meningitis.

The term “recombinant” as used herein shall mean “being prepared by or the result of genetic engineering”. A recombinant host specifically comprises an expression vector or cloning vector, or it has been genetically engineered to contain a recombinant nucleic acid sequence, in particular employing nucleotide sequence foreign to the host. A recombinant protein is produced by expressing a respective recombinant nucleic acid in a host. The term “recombinant antibody”, as used herein, includes antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody e.g., from a transfectoma, (c) antibodies isolated from a recombinant, combinatorial human antibody library or library of antigen-binding sequences of an antibody, and (d) antibodies prepared, expressed, created or isolated by any other means that involve splicing of human immunoglobulin gene sequences to other DNA sequences. Such recombinant antibodies comprise antibodies engineered to include rearrangements and mutations which occur, for example, during antibody maturation. In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual, Cold Spring Harbor, (1982).

Selective binding can be further improved by recombinant antibody optimization methods known in the art. For example, certain regions of the variable regions of the immunoglobulin chains described herein may be subjected to one or more optimization strategies, including light chain shuffling, destinational mutagenesis, CDR amalgamation, and directed mutagenesis of selected CDR and/or framework regions.

The term “subject” as used herein shall refer to a warm-blooded mammalian, particularly a human being or a non-human animal. K. pneumoniae is a critically important human pathogen that is also an emerging concern in veterinary medicine. It is present in a wide range of non-human animal species. Thus, the term “subject” may also particularly refer to animals including dogs, cats, rabbits, horses, cattle, pigs and poultry. In particular the medical use of the invention or the respective method of treatment applies to a subject in need of prophylaxis or treatment of a disease condition associated with a K. pneumoniae infection. The subject may be a patient at risk of a K. pneumoniae infection or suffering from disease, including early stage or late stage disease. The term “patient” includes human and other mammalian subjects that receive either prophylactic or therapeutic treatment. The term “treatment” is thus meant to include both prophylactic and therapeutic treatment.

A subject is e.g., treated for prophylaxis or therapy of K. pneumoniae disease conditions. In particular, the subject is treated, which is either at risk of infection or developing such disease or disease recurrence, or a subject that is suffering from such infection and/or disease associated with such infection.

Specifically the term “prophylaxis” refers to preventive measures which is intended to encompass prevention of the onset of pathogenesis or prophylactic measures to reduce the risk of pathogenesis.

Specifically, the treatment may be by interfering with the pathogenesis of K. pneumoniae as causal agent of the condition,

The term “substantially pure” or “purified” as used herein shall refer to a preparation comprising at least 50% (w/w), preferably at least 60%, 70%, 80%, 90% or 95% of a compound, such as a nucleic acid molecule or an antibody. Purity is measured by methods appropriate for the compound (e.g., chromatographic methods, polyacrylamide gel electrophoresis, HPLC analysis, and the like).

The term “therapeutically effective amount”, used herein interchangeably with any of the terms “effective amount” or “sufficient amount” of a compound e.g., an antibody of the present invention, is a quantity or activity sufficient to, when administered to the subject effect beneficial or desired results, including clinical results, and, as such, an effective amount or synonym thereof depends upon the context in which it is being applied.

An effective amount is intended to mean that amount of a compound that is sufficient to treat, prevent or inhibit such diseases or disorder. In the context of disease, therapeutically effective amounts of the antibody as described herein are specifically used to treat, modulate, attenuate, reverse, or affect a disease or condition that benefits from an inhibition of K. pneumoniae pathogenesis, for example, adhesion and colonization of mucosal surfaces, uncontrolled replication within sterile body sites, and toxicity of host cells by bacterial products.

The amount of the compound that will correspond to such an effective amount will vary depending on various factors, such as the given drug or compound, the pharmaceutical formulation, the route of administration, the type of disease or disorder, the identity of the subject or host being treated, and the like, but can nevertheless be routinely determined by one skilled in the art.

A therapeutically effective amount of the antibody as described herein, such as provided to a human patient in need thereof, may specifically be in the range of 0.5-50 mg/kg, preferably 5-40 mg/kg, even more preferred up to 20 mg/kg, up to 10 mg/kg, up to 5 mg/kg, though higher doses may be indicated e.g., for treating acute disease conditions. The dose can be much lower if a highly potent antibody is used. In such case, the effective amount may be in the range of 0.005 to 5 mg/kg, preferably 0.05 to 1 mg/kg.

Moreover, a treatment or prevention regime of a subject with a therapeutically effective amount of the antibody of the present invention may consist of a single administration, or alternatively comprise a series of applications. For example, the antibody may be administered at least once a year, at least once a half-year or at least once a month. However, in another embodiment, the antibody may be administered to the subject from about one time per week to about a daily administration for a given treatment. The length of the treatment period depends on a variety of factors, such as the severity of the disease, either acute or chronic disease, the age of the patient, the concentration and the activity of the antibody format. It will also be appreciated that the effective dosage used for the treatment or prophylaxis may increase or decrease over the course of a particular treatment or prophylaxis regime. Changes in dosage may result and become apparent by standard diagnostic assays known in the art. In some instances, chronic administration may be required.

LPS neutralizing and bactericidal mAbs highly specific to the O1 antigen of K. pneumoniae have great potential for the prophylaxis (e.g., for high risk groups) and treatment of K. pneumoniae infections. Doses for prophylactic treatment are typically in the lower range (e.g. at least 0.005 mg/kg and less than 1 mg/kg), and specifically administered once, e.g. when a subject is identified as being immunocompromised or immunosuppressed and/or at risk of getting in contact with K. pneumoniae, or by a long-term treatment schedule, e.g. at least 2, 3, 4, 5, 6, 7, 8, 9, 10, 11, or 12 doses annually or half-annually. Doses for therapeutic treatment are typically administered in the acute or chronic phase of disease and typically in the higher range (e.g. at least 0.05 or 0.5 mg/kg and less than 10 mg/kg), and specifically administered until cure of the disease, by one or more administrations, e.g. in regular intervals, such as at least 1, 2, 3, or 4 administrations daily, or at least 1, 2, 3, 4, 5, or 6 administrations weekly, or at least 1, 2, 3, or 4 administrations monthly.

Aiming to develop monoclonal antibodies for the prevention and treatment of infections caused by Klebsiella strains, the molecular target of specific mAbs suitably is the LPS O-antigen, which shows limited heterogeneity in Klebsiella. Such O-side chain is considered immunorelevant because not fully masked by bulky capsular polysaccharide.

Once antibodies with the desired binding properties are identified, such antibodies, including antibody fragments can be produced by methods well-known in the art, including, for example, hybridoma techniques or recombinant DNA technology.

Recombinant monoclonal antibodies can, for example, be produced by isolating the DNA encoding the required antibody chains and transfecting a recombinant host cell with the coding sequences for expression, using well known recombinant expression vectors, e.g., the plasmids of the invention or expression cassette(s) comprising the nucleotide sequences encoding the antibody sequences. Recombinant host cells can be prokaryotic and eukaryotic cells, such as those described above.

According to a specific aspect, the nucleotide sequence may be used for genetic manipulation to obtain antibodies containing artificial sequences, e.g. to improve the affinity, or other characteristics of the antibody. For example, the constant region may be engineered to more nearly resemble human constant regions to avoid immune response, if the antibody is used in clinical trials and treatments in humans. It may be desirable to genetically manipulate the antibody sequence to obtain greater affinity to the 01 target and greater efficacy against Klebsiella pneumoniae. It will be apparent to one of skill in the art that one or more polynucleotide changes can be made to the antibody and still maintain its binding ability to the target O1 antigen.

The production of antibody molecules, by various means, is generally well understood. U.S. Pat. No. 6,331,415 (Cabilly et al.), for example, describes a method for the recombinant production of antibodies where the heavy and light chains are expressed simultaneously from a single vector or from two separate vectors in a single cell. Wibbenmeyer et al., (1999, Biochim Biophys Acta 1430(2):191-202) and Lee and Kwak (2003, J. Biotechnology 101:189-198) describe the production of monoclonal antibodies from separately produced heavy and light chains, using plasmids expressed in separate cultures of host cells. Various other techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988).

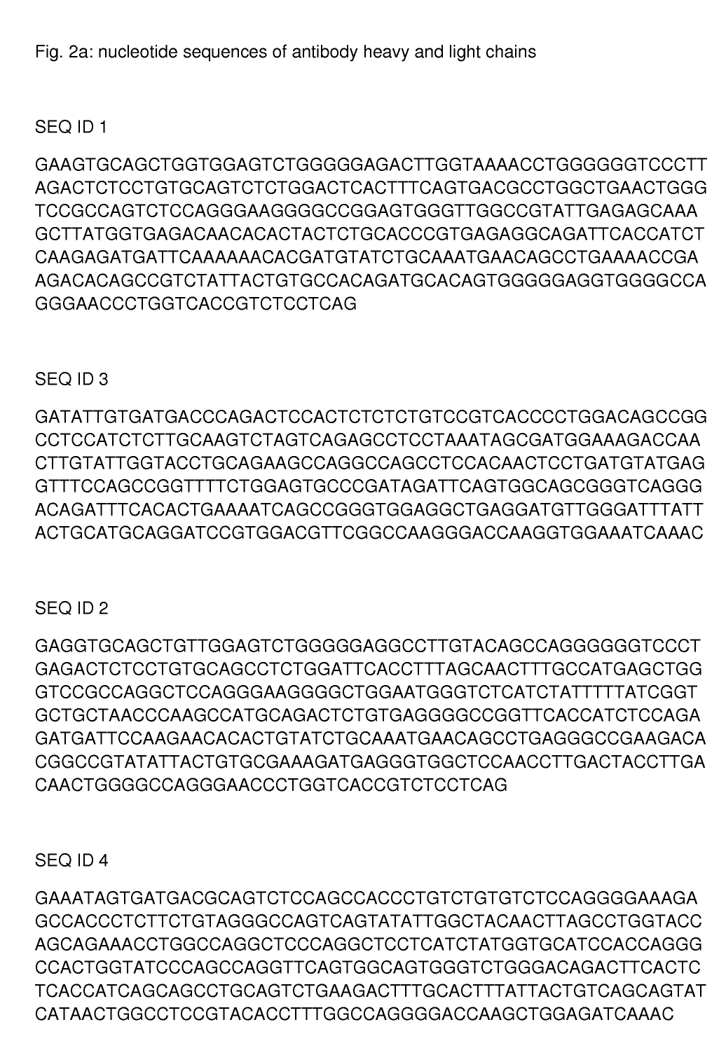

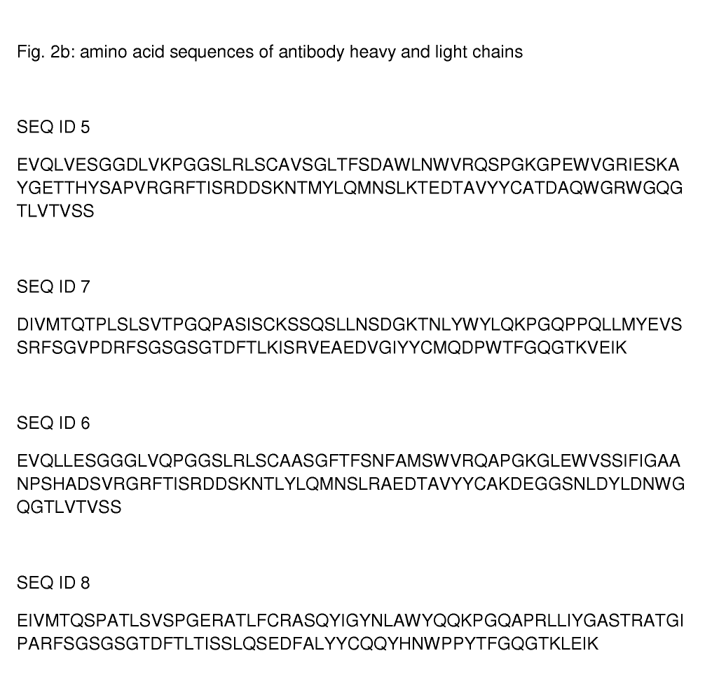

If desired, the antibody as described herein, e.g., any of the mAbs referred to in the examples or listed in FIG. 1 or 2, or a functional variant thereof, may be sequenced and the polynucleotide sequence may then be cloned into a vector for expression or propagation. The sequence encoding the antibody may be maintained in vector in a host cell and the host cell can then be expanded and frozen for future use. Production of recombinant monoclonal antibodies in cell culture can be carried out through cloning of antibody genes from B cells by means known in the art.

In another aspect, there is provided an isolated nucleic acid comprising a sequence that codes for production of the recombinant antibody as described herein.

An antibody encoding nucleic acid can have any suitable characteristics and comprise any suitable features or combinations thereof. Thus, for example, an antibody encoding nucleic acid may be in the form of DNA, RNA, or a hybrid thereof, and may include non-naturally-occurring bases, a modified backbone, e.g., a phosphorothioate backbone that promotes stability of the nucleic acid, or both. The nucleic acid advantageously may be incorporated in an expression cassette, vector or plasmid of the invention, comprising features that promote desired expression, replication, and/or selection in target host cell(s). Examples of such features include an origin of replication component, a selection gene component, a promoter component, an enhancer element component, a polyadenylation sequence component, a termination component, and the like, numerous suitable examples of which are known.

The present disclosure further provides the recombinant DNA constructs comprising one or more of the nucleotide sequences described herein. These recombinant constructs are used in connection with a vector, such as a plasmid, phagemid, phage or viral vector, into which a DNA molecule encoding any disclosed antibody is inserted.

Monoclonal antibodies are produced using any method that produces antibody molecules by cell lines in culture e.g., cultivating recombinant eukaryotic (mammalian or insect) or prokaryotic (bacterial) host cells. Examples of suitable methods for preparing monoclonal antibodies include the hybridoma methods of Kohler et al. (1975, Nature 256:495-497) and the human B-cell hybridoma method (Kozbor, 1984, J. Immunol. 133:3001; and Brodeur et al., 1987, Monoclonal Antibody Production Techniques and Applications, (Marcel Dekker, Inc., New York), pp. 51-63).

Antibodies as described herein may be identified or obtained employing a hybridoma method or by direct amplification, cloning and recombinant expression of immunoglobulin genes from single B cells including e.g. a screening method as exemplified herein using a certain antigen. In such hybridoma method, a mouse or other appropriate host animal, such as a hamster, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell.

Culture medium in which hybridoma cells or of cells producing recombinant antibodies are growing is assayed for production of monoclonal antibodies directed against the antigen. Preferably, the binding specificity of monoclonal antibodies produced by hybridoma cells or by cells producing the antibody recombinantly is determined by immunoprecipitation or by an in vitro binding assay, such as radioimmunoassay (RIA) or enzyme-linked immunoabsorbent assay (ELISA).

mAbs may then be purified from hybridoma supernatants and culture supernatants from cells producing recombinant antibodies for further testing for its specific binding of the O1 antigen and antibodies may be engineered e.g., for different diagnostic or therapeutic purposes.

Gal-II specific antibodies, in some instances, emerge through screening against the single gal-II antigen. To increase the likelihood of isolating differentially binding clones one would apply multiple selective pressures by processively screening against the different antigens.

Screening methods for identifying antibodies with the desired selective binding properties may be done by display technologies using a library displaying antibody sequences or antigen-binding sequences thereof (e.g. using phage, bacterial, yeast or mammalian cells; or in vitro display systems translating nucleic acid information into respective (poly)peptides). Reactivity can be assessed based on ELISA, Immunoblotting or surface staining with flow cytometry, e.g. using standard assays.

Isolated antigen(s) may e.g. be used for selecting antibodies from an antibody library, e.g. a yeast-displayed antibody library.

For example, the invention specifically provides for gal-II specific antibodies, which are obtained by a process to identify antibodies with specificities to bind the gal-II antigen, e.g. by a specific discovery selection scheme. Accordingly, an antibody library including antibodies showing reactivity with the gal-II target, may be selected for reactivity with the target.

The invention moreover provides a pharmaceutical composition which comprises an antibody as described herein and a pharmaceutically acceptable carrier or excipient. These pharmaceutical compositions can be administered in accordance with the present invention as a bolus injection or infusion or by continuous infusion. Pharmaceutical carriers suitable for facilitating such means of administration are well known in the art.

Pharmaceutically acceptable carriers generally include any and all suitable solvents, dispersion media, coatings, antibacterial and antifungal agents, isotonic and absorption delaying agents, and the like that are physiologically compatible with an antibody or related composition or combination provided by the invention. Further examples of pharmaceutically acceptable carriers include sterile water, saline, phosphate buffered saline, dextrose, glycerol, ethanol, and the like, as well as combinations of any thereof.

In one such aspect, an antibody can be combined with one or more carriers appropriate a desired route of administration, antibodies may be e.g., admixed with any of lactose, sucrose, starch, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, polyvinyl alcohol, and optionally further tableted or encapsulated for conventional administration. Alternatively, an antibody may be dissolved in saline, water, polyethylene glycol, propylene glycol, carboxymethyl cellulose colloidal solutions, ethanol, corn oil, peanut oil, cotton-seed oil, sesame oil, tragacanth gum, and/or various buffers. Other carriers, adjuvants, and modes of administration are well known in the pharmaceutical arts. A carrier may include a controlled release material or time delay material, such as glyceryl monostearate or glyceryl distearate alone or with a wax, or other materials well known in the art.

Additional pharmaceutically acceptable carriers are known in the art and described in, e.g., REMINGTON'S PHARMACEUTICAL SCIENCES. Liquid formulations can be solutions, emulsions or suspensions and can include excipients such as suspending agents, solubilizers, surfactants, preservatives, and chelating agents.

Pharmaceutical compositions are contemplated wherein an antibody as described herein and one or more therapeutically active agents are formulated. Stable formulations of the antibody as described herein are prepared for storage by mixing said immunoglobulin having the desired degree of purity with optional pharmaceutically acceptable carriers, excipients or stabilizers, in the form of lyophilized formulations or aqueous solutions. The formulations to be used for in vivo administration are specifically sterile, preferably in the form of a sterile aqueous solution. This is readily accomplished by filtration through sterile filtration membranes or other methods. The antibody and other therapeutically active agents disclosed herein may also be formulated as immunoliposomes, and/or entrapped in microcapsules.

Administration of the pharmaceutical composition comprising an antibody as described herein, may be done in a variety of ways, including orally, subcutaneously, intravenously, intranasally, intraotically, transdermally, mucosal, topically, e.g., gels, salves, lotions, creams, etc., intraperitoneally, intramuscularly, intrapulmonary e.g., employing inhalable technology or pulmonary delivery systems, vaginally, parenterally, rectally, or intraocularly.

Examplary formulations as used for parenteral administration include those suitable for subcutaneous, intramuscular or intravenous injection as, for example, a sterile solution, emulsion or suspension.

In one embodiment, the antibody as described herein is the only therapeutically active agent administered to a subject e.g., as a disease modifying or preventing monotherapy.

In another embodiment, the antibody as described herein is combined with further antibodies in a cocktail e.g., combined in a mixture or kit of parts, to target Klebsiella pneumoniae, such that the cocktail contains more than one therapeutically active agents administered to a subject e.g., as a disease modifying or preventing combination therapy.

Further, the antibody as described herein may be administered in combination with one or more other therapeutic or prophylactic agents, including but not limited to standard treatment e.g., antibiotics, steroid and non-steroid inhibitors of inflammation, and/or other antibody based therapy e.g., employing anti-bacterial or anti-inflammatory agents.

A combination therapy is particularly employing a standard regimen e.g., as used for treating infection by Klebsiella pneumoniae. This may include antibiotics, e.g., tygecycline, colistin, polymixin B, and beta lactams with or without non-beta lactam inhibitors.

In a combination therapy, the antibody may be administered as a mixture, or concomitantly with one or more other therapeutic regimens e.g., either before, simultaneously or after concomitant therapy.

The biological properties of the antibody or the respective pharmaceutical preparations as described herein may be characterized ex vivo in cell, tissue, and whole organism experiments. As is known in the art, drugs are often tested in vivo in animals, including but not limited to mice, rats, rabbits, dogs, cats, pigs, and monkeys, in order to measure a drug's efficacy for treatment against a disease or disease model, or to measure a drug's pharmacokinetics, pharmacodynamics, toxicity, and other properties. The animals may be referred to as disease models. Therapeutics are often tested in mice, including but not limited to nude mice, SCID mice, xenograft mice, and transgenic mice (including knockins and knockouts). Such experimentation may provide meaningful data for determination of the potential of the antibody to be used as a therapeutic or as a prophylactic with the appropriate half-life, effector function, (cross-) neutralizing activity and/or immune response upon active or passive immunotherapy. Any organism, preferably mammals, may be used for testing. For example because of their genetic similarity to humans, primates, monkeys can be suitable therapeutic models, and thus may be used to test the efficacy, toxicity, pharmacokinetics, pharmacodynamics, half-life, or other property of the subject agent or composition. Tests in humans are ultimately required for approval as drugs, and thus of course these experiments are contemplated. Thus, the antibody and respective pharmaceutical compositions as described herein may be tested in humans to determine their therapeutic or prophylactic efficacy, toxicity, immunogenicity, pharmacokinetics, and/or other clinical properties.

In specific cases the patient is an immunocompromised patient. Some immunocompromised patients may suffer from a primary immunodeficiency or a secondary (acquired) immunodeficiency. Some immunocompromised patients are being or have been treated with an immunosuppressive therapy or with a chemotherapeutic agent. Some immunocompromised patients are transplant patient.

Immunocompromised patients likely suffer from a phagocytic disorder, such as characterized by a lower phagycytic number and/or impaired function.

The following disorders can cause impaired or lost phagocytotic activities:

Primary immunodeficiency of phagocytes:

1. Chronic neutropenia:
a. Cyclic neutropenia
b. Severe congenital neutropenia
c. Shwachman-Diamond syndrome

2. Leukocyte adhesion deficiency
a. Type 1
b. Type 2
c. Rac 2 deficiency

3. Defects of signaling
a. Interferon-γ and interleukin-12 defects

4. Defects of intracellular killing
a. Chronic granulomatous disease of childhood
b. Myeloperoxidase deficiency
c. Chédiak-Higashi syndrome
d. Neutrophil-specific granule deficiency

Secondary immunodeficiency of phagocytes:

1. Neutropenia/granulocytopenia: reduced number of blood neutrophils/granulocytes (<1500 cells/ml)
a. Bone marrow diseases (tumor infiltration, aplastic anaemia, hematologic malignancy, granulomatous disease, irradiation, myelofibrosis)
b. Immune mediated neutropenia (drugs acting as hapten, autoimmune diseases)
c. Infections (bacterial sepsis, malaria, toxoplasmosis, viral infections, like EBV, CMV, Influenza)
d. Nutritional deficiency (malnutrition, B-12 deficiency)
e. Drugs, chemicals (macrolids, procainamides, phenotiazid, sulfonamides, chloramphenicol, aminopyrine, anti-thyroid drugs, like thiouracil, methimazol, thiocyanate, heavy metals)
f. Chemotherapy, immunosuppression (treatment of autoimmune diseases, after transplantation)

2. Phagocyte function/chemotaxis disorder or decreased ability to upregulate production of phagocytes
a. Neonates (Under conditions of stress, neonatal PMNs do not function with normal phagocytic and microbicidal activities. PMNs isolated from the blood of term neonates display diminished chemotactic and adhesion capacities.
b. Elderly (Decreased phagocytic ability, cytotoxicity, enzyme release, reduced adhesion
c. Diabetes mellitus (lower killing by PMNs, monocyte/macrophage dysfunction, renal failure and cirrhosis
d. Trisomy 21
e. Surgery, trauma
f. Corticosteroids
g. HIV

To identify patients with impaired phagocyte number and function, any suitable technique known by persons skilled in the art can be applied. These include but are not limited to complete blood count, differential white blood cell count, peripheral smear, measurement of adherence, chemotaxis, phagocytosis, intracellular killing of phagocytes, assays to measure specific neutrophil enzymes or detect autoantibodies against neutrophils.

The present invention is further illustrated by the following examples without being limited thereto.

### EXAMPLES

### Example 1

### Example 1: Generation of Fully Human Anti-Klebsiella pneumoniae Lipopolysaccharide O1-Antigen Antibodies from Peripheral Memory and Intestinal Effector B Cells

Methods

1. Isolation and generation of recombinant fully human Klebsiella pneumoniae O-antigen-binding antibodies

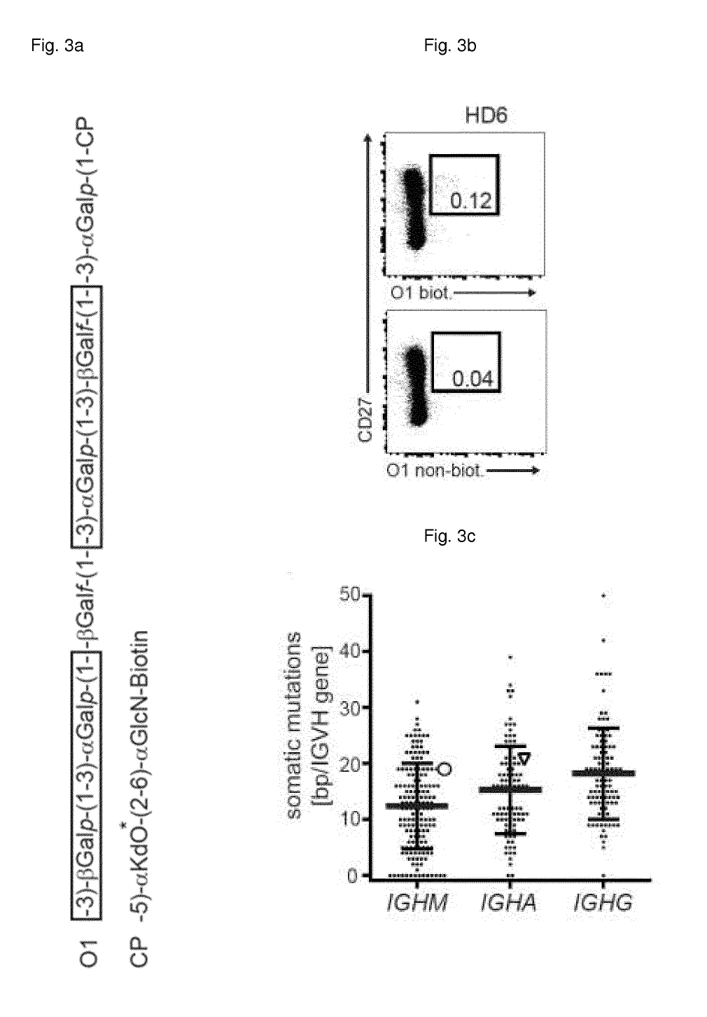

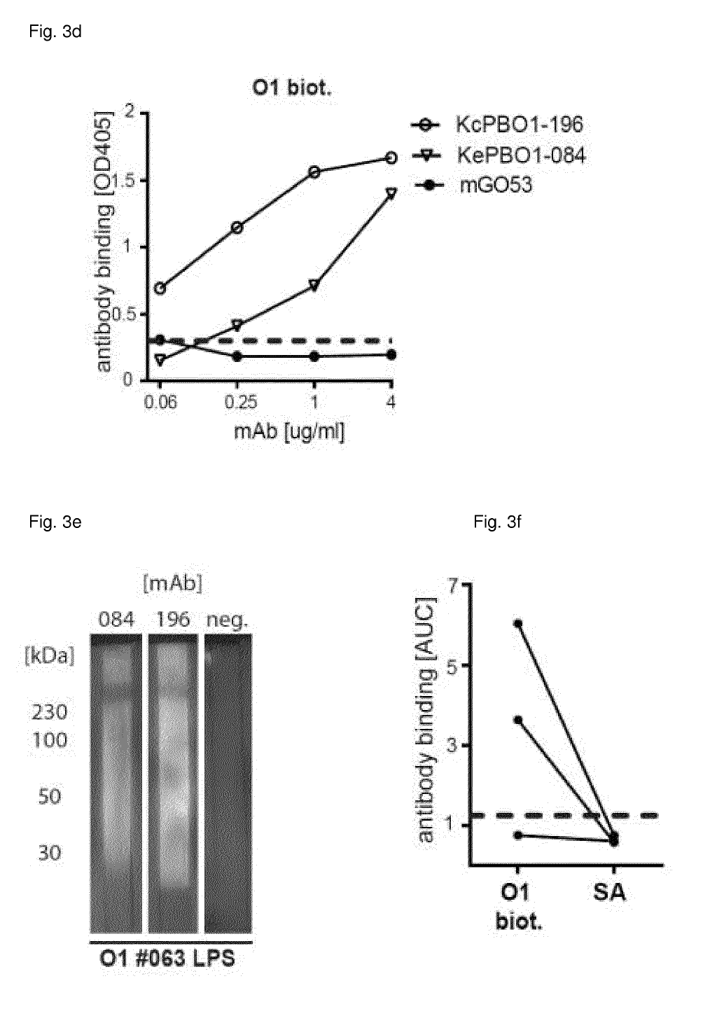

O-antigen-binding B cells were identified by flow cytometry using fluorophore-conjugated Streptavidin to detect biotinylated O1-antigen, as shown in FIG. 3a, bound by B cells from human peripheral blood.

Subsequently, single O-antigen-binding B cells were isolated using fluorescence-activated cell sorting (FIG. 3b).

1.1 Isolation of Peripheral Blood Mononuclear Cells

Freshly drawn human peripheral blood was diluted 1:1 with RPMI medium (Gibco) at room-temperature and slowly added onto 15 ml Ficoll (GE Healthcare) in a 50 ml centrifuge tube. The cells were spun for 40 min at room-temperature with the lowest acceleration and no break. Cells residing at the water/Ficoll interface were isolated using a Pasteur pipet and resuspended in a minimum of 25 ml RPMI at room-temperature in a 50 ml centrifuge tube. All subsequent steps were performed at 4° C. and on ice. Cells were centrifuged at 400 g for 10 min. The supernatant was discarded and the cells were washed in 10 ml ice-cold RPMI. The cells were counterstained using Trypan Blue (Gibco), counted using a Thoma chamber and subsequently centrifuged at 400 g for 10 min. The supernatant was discarded and cells were further processed for flow cytometry or immediately frozen following the freezing protocol.

1.2 Isolation of Lamina Propria Lymphocytes

As an alternative source, lamina propria lymphocytes (LPL) can be used, which are e.g., directly isolated from human terminal ileum surgical samples. According to a typical protocol, all cells are isolated from phenotypical healthy mucosa having at least 3 cm distance from tumor or inflamed area. Lamina mucosa and propria are dissected from lamina muscularis using forceps and scalpel. The tissue is extensively washed in PBS+(1×PBS (Gibco), 2% FCS, 1× Antimycotic/Antibiotic (Gibco)) at room-temperature. The tissue is kept on ice throughout the process, except if otherwise stated. The tissue is cut into 3-5 mm pieces and remaining connective tissue is removed as extensively as possible. The tissue is transferred to a 50 ml centrifuge tube and washed 3-times with 1×PBS+ and subsequently incubated 2×15 min with PBS containing 1 mM Dithioerythriol in a bottle placed in a water bath at 37° C. under constant stirring to remove residual mucus. Subsequently the tissue is washed 3× with 1×PBS containing 0.5 mM EDTA, followed by 30 min incubation with 1×PBS containing 0.5 mM EDTA at 37° C. as described above to remove the epithelium. After washing with 1×PBS+ the tissue is digested using 1×PBS+ containing 0.2% (w/v) Dnasel and 0.5% (w/v) Collagenase D (both Roche) for 1 h under constant stirring at 37° C. LPL are isolated by a discontinuous Percoll gradient (40%/70% diluted in 1×PBS). To better discriminate the 40%/70% interface Phenol Red is added to the 70% dilution (1:1000; Gibco). 15 ml of each dilution are added into a 50 ml centrifuge tube and 20 ml cell suspension is slowly added onto the top of the gradient. After centrifugation, LPL are isolated from the 40%/70% interface by using a Pasteur pipet and added into a minimum of 25 ml RPMI into a new centrifuge tube. All subsequent steps are performed at 4° C. and on ice. Cells are centrifuged at 400 g for 10 min. The supernatant is discarded and the cells are washed in 10 ml ice-cold RPMI. The cells are counted using a Thoma chamber using a Trypan Blue counterstain (Gibco) and subsequently centrifuged at 400 g for 10 min. The supernatant is discarded and cells are further processed for flow cytometry or immediately frozen following the freezing protocol.

1.3 Freezing of Mammalian Cells

Cells were counted and diluted with heat-inactivated FCS (Gibco) to reach a concentration of 1×10⁷ cells/ml. FCS containing 20% (v/v) sterile DMSO suitable for cell culture (Sigma) was freshly prepared and 500 μl were added to 1.8 ml cryotubes (ThermoFisher). 500 μl cell suspension was added to reach a final concentration of 5×10⁶ cells/ml and the vials were frozen at −80° C. using a Coolcell (Biocision).

1.4 Cell Staining for Flow Cytometry

Flow cytometry cell stainings were performed in 1.5 ml tubes using 1×PBS containing 2% FCS (FACS buffer) or Horizon stain buffer (BD) if more than one Brilliant violet dye was used. 5×10⁶ Cells/ml were stained in 50 μl staining mix for 30 min at 4° C. in the dark using the following antibodies:

Mouse anti-human CD19-APC-H7 (BD), Mouse anti-human CD27-PE (BD), Mouse anti-human CD27-BV605 (BD), Mouse anti-human IgG BV510 (BD), Mouse anti-human IgG V450 (BD), Mouse anti-human IgA-PE (Miltenyi), Goat anti-human IgA-FITC (Life technologies), Mouse anti-human CD45-VioGreen (Miltenyi) and Mouse anti-human CD11b-PE-Cy7 (eBioscience). Dead cells were excluded by 7-AAD (Life technologies). Biotinylated O-antigen fractions were used at a final concentration of 20 μg/ml and detected with 0.5 μg/ml Streptavidin-Alexa647 (Life technologies). Subsequently, 1 ml FACS buffer was added and the cells were spun for 5 min at 500 g at 4° C. The supernatant was discarded and the cells were washed in 1 ml FACS buffer and centrifuged again. The pellet was resuspended in 50 μl staining mix and cells were incubated 30 min at 4° C. in the dark. For washing 1 ml of FACS buffer was added before the centrifugation step. Supernatant was discarded and cells were resuspended in 1 ml FACS buffer prior to centrifugation. After supernatant removal the cells were diluted using FACS buffer (250-1000 μl) and filtered into a tube with a meshed cap (BD) before analysis or sorting.

1.5 Single O-antigen binding B cell sorting

O1-antigen binding memory B cells were identified as single, 7-AAD⁻, CD19⁺, CD27⁺, O-antigen⁺ cells. Single cells were sorted on Aria II instruments (BD) into 384-well PCR plates (4titude). After single cell sorting the plates were immediately frozen on dry ice and stored at −80° C. or directly processed.

1.6 Ig Gene Sequencing of Single Human B Cells

Full length Ig genes of single human B cells were obtained by the method described by Tiller et al modified by Murugan et al. (T. Tiller et al., Efficient generation of monoclonal antibodies from single human B cells by single cell RT-PCR and expression vector cloning. J. Immunol. Methods. 329, 112-24 (2008); R. Murugan, K. Imkeller, C. E. Busse, H. Wardemann, Direct high-throughput amplification and sequencing of immunoglobulin genes from single human B cells. Eur. J. Immunol. 45, 2698-700 (2015)). Full cDNA of single B cells was synthesized by reverse transcription in a 384-well cycler (Eppendorf). cDNA was transferred to a primary 384-well PCR plate. After the primary amplification step, the primary PCR product was transferred into secondary PCR mix. Primers used to amplify the full length Ig genes were previously published by others. (T. Tiller et al., Efficient generation of monoclonal antibodies from single human B cells by single cell RT-PCR and expression vector cloning. J. Immunol. Methods. 329, 112-24 (2008), R. Murugan, K. Imkeller, C. E. Busse, H. Wardemann, Direct high-throughput amplification and sequencing of immunoglobulin genes from single human B cells. Eur. J. Immunol. 45, 2698-700 (2015); H. Wardemann et al., Predominant autoantibody production by early human B cell precursors. Science. 301, 1374-7 (2003); J. Benckert et al., The majority of intestinal IgA+ and IgG+ plasmablasts in the human gut are antigen-specific. J. Clin. Invest. 121, 1946-55 (2011)). Sequence information was obtained by Sanger sequencing.

1.7 Cloning of Recombinant Fully Human Klebsiella pneumoniae O-Antigen Antibodies

In order to produce fully human antibody, the IgHeavy and the corresponding IgLight genes were first cloned into human Igγ1 and Igκ or Igλ expression vectors, respectively. Therefore, the IgHeavy and the corresponding IgLight genes were specifically amplified from the primary PCR product using V-segment and J-segment specific primers containing appropriate restriction sites. The Ig gene PCR fragments were purified and digested using the respective restriction enzymes. Afterwards the Specific PCR Ig gene fragments were ligated into human Igγ1 and Igκ or Igλ expression vectors containing the respective human Ig constant region. The Igγ1 expression vector was equipped with secretory splice variant of the Ig constant, enabling the secretion of antibody into the cell culture supernatant.

In order to amplify successfully ligated expression vectors, the vectors were transformed into chemically competent E. coli (DH10B, Invitrogen). To select positive clones the whole solution were plated on LB plates containing 100 μg/ml Ampicillin and incubated for a minimum of 16 h at 37° C. To confirm correct insertion into the respective expression vector, we performed Insert check PCR on bacterial colonies using appropriate primer pairs and sent the product for purification and sequencing by Sanger sequencing (Eurofins genomics). First, the obtained sequence was checked for in-frame insertion of the respective Ig gene. Afterwards, the sequence was compared to the secondary PCR product sequence and excluded if PCR-prone additional point mutations in the Insert check PCR sequence were found. If point mutations found in the secondary PCR product were not present in the Insert-check PCR sequence, these mutations were not included into the analysis, due to a high likelihood that these mutations were generated early in the secondary PCR process. To amplify correctly cloned expression vectors, bacteria bearing the correct plasmid were inoculated into 4 ml TB (Gibco) containing 75₄/ml Ampicillin in 13 ml culture tubes (Sarstedt) and grown for a minimum of 16 h at 37° C. at 180 rpm. The plasmid DNA was extracted using the Nucleospin Kit (Macherey & Nagel) according to the manufacturer's instructions.

1.8 Expression of Recombinant Fully Human Klebsiella pneumoniae O1-Antigen Antibodies

The fully human IgG1 antibodies were produced by Polyethylenimine—(PEI) mediated transfection of adherent and non-adherent human embryonic kidney 293 cells, HEK293T or HEK293S (Invitrogen), respectively.

1.8.1 Mammalian Cell Culture

HEK293T were cultured at 37° C. in 5% CO₂ in 25 ml DMEM GlutaMAX media containing 10% (v/v) FCS and 1× Antibiotic/Antimycotic (Gibco), whereas HEK293S cells were cultured at 37° C. in 5% CO₂ in 20 ml Freestyle medium (Gibco) at 180 rpm in 50 ml Bioreactors (TPP).

1.8.2 PEI-Mediated Transfection of HEK293T Cells

The cationic polymer PEI was used for transient gene transfer to HEK293T cells. Therefore, 10-15 μg IgH vector was mixed with equal amount of its corresponding IgL vector and 50 μl/μg total DNA of 150 mM sterile sodium chloride solution was added. Subsequently PEI [0.6 mg/ml] was added in a 3:1 (w/w) DNA to PEI ratio. The solution was immediately vortexed for 10 s and incubated at room-temperature for 10 min. In the meantime, plates were washed with 10 ml DMEM Glutamax pre-warmed to 37° C. to remove residual bovine serum antibodies. Thus, 25 ml pre-warmed expression media (DMEM Glutamax containing 1× Antibiotic/Antimycotic (Gibco) and 1× serum-free media supplement Nutridoma (Roche) was added and the cells were incubated at 37° C. in 5% CO₂ until further use. Hence, the transfection mix was added drop-wise to the cells and the cells were incubated for 3.5 days. Subsequently, the antibody secreted into the supernatant was harvested and the cells were again incubated with 25 ml expression media. The supernatants were centrifuged at 4000 g to remove cell debris and transferred into a sterile 50 ml centrifuge tube (Sarstedt).

1.8.3 PEI-Mediated Transfection of HEK293S Cells

HEK293S cells were transiently transfected using the cationic polymer PEI. 10 ml HEK293S cells were seeded at 1.5×10⁶ Cells/ml in Freestyle 293 Expression medium the day before transfection. After 16 h, the cell number was determined to be approximately 2.5×10⁶ Cells/ml using a Thoma chamber. Thus, 10-15 μg IgH vector was mixed with equal amount of its corresponding IgL vector and added to the cell suspension. Subsequently cells were incubated an additional 5 min. To transfect the prepared cells PEI [0.6 mg/ml] was added in a 3:1 (w/w) DNA to PEI ratio. After 24 h 10 ml Ex-Cell medium (Gibco) containing 4 mM L-Glutamine (Gibco) was added to the cells and incubate for 5 days at 37° C. in 5% CO₂. The supernatants were centrifuged at 4000 g to remove cell debris and transferred into a sterile 50 ml centrifuge tube (Sarstedt).

1.9 Antibody Purification

In order to purify the secreted antibody from cell culture supernatant 12.5 μl Protein-G-coupled beads (GE Healthcare) per 10 ml antibody containing supernatant were washed with 50 ml ice-cold sterile 1×PBS pH=7.4 (Gibco) by centrifugation at 4000 g 4° C. for 10 min. The supernatant was carefully removed from the beads and an appropriate volume of 100 μl/sample was left in the centrifugation tube and added to the antibody supernatants. The mixture was incubated for at least 12 h at 4° C. on a rotator. Hence, the beads were harvested by centrifugation at 4000 g 4° C. for 10 min and the supernatant was carefully removed and added into a new sterile 50 ml centrifugation tube if needed. The beads were added onto a chromatography column (Bio-Rad) which has been equilibrated with 2 ml of ice-cold PBS. The columns were emptied by gravity-flow or by applying pressure with the thumb. Beads were washed with 1.5 ml ice-cold PBS. Thus, antibody was released from Protein-G into a 1.5 ml tube by a low pH pulse applying 450 μl sterile 0.1 M Glycine pH=3 for 3 min and the solution was buffered by adding a 1:10 equivalent of a sterile 1M Tris solution pH=8. The procedure was repeated using 225 μl Glycine solution and eluted into a second sterile 1.5 ml tube. The pH=7.4-8.0 of the solution was confirmed by adding approximately 10 μl solution onto a small pH indicator strip (Sigma).

1.10 Antibody Concentration Measurement

Antibody concentrations in purified fractions were measured by Enzyme-linked immunosorbent assay (ELISA). Therefore a 96-well high-binding plate (Costar) was coated with 50 μl 1:500 dilution of a goat anti-human IgG Fcγ-fragment specific capture antibody (Dianova) for at least 12 h at 4° C. Thus, the plates were washed 3-times with deionized water and 200 μl blocking buffer (1×PBS, 0.05% Tween 20 and 1 mM EDTA) was added per well for 1 h. After additional 3-times washing, the plates were incubated with eight 50 μl 1:2.5 serial antibody dilutions in PBS and incubated for 1 h. Two serial dilutions of human IgG from human plasma (Sigma) starting with 1 μg/ml and 3 μg/ml served as a standard. After washing, 50 μl of a 1:1000 HRP-coupled goat anti-human IgG secondary antibody was added for 1 h. After an additional washing step, 1000 HRP ABTS substrate was added and the amount of bound antibody was detected as the optical density at 405 nm.

2. Antibody Reactivity Measurement

Purified monoclonal antibodies were tested for binding to K. pneumoniae LPS O1 O-antigen by ELISA or ImmunoBlot.

2.1 O-Antigen ELISA

In order to immobilize biotinylated O-antigen samples, high-binding 96-well ELISA Plates (Costar) were coated overnight with 50 ul of 1 ug/ml Streptavidin (NEB) in PBS. Subsequently, plates were washed 3-times in PBS before adding 50 μl of either 2 μg/ml of biotinylated O-polysaccharide was generated from purified LPS of Klebsiella pneumoniae serotype O1 (strain ATCC43816, O1 antigen characterized by a structure shown in FIG. 3a).

After incubation for 1 h at room-temperature, plates were washed and incubated with 200 μl 2% BSA in PBS for 1 h at room-temperature. After washing, 1:4 serial dilutions of recombinant human IgG1 antibodies with a starting concentration of 4 μg/ml were added to the plate for 1 h at room-temperature. After an additional washing step, concentration-dependent binding was detected using 50 μl:1000 goat anti-human IgG Fc HRP-coupled (Jackson) secondary antibody diluted in blocking buffer. After washing, 100 μl HRP ABTS substrate was added and antibody binding was detected as optical density at 405 nm. A non-polyreactive mature naïve antibody, which has been previously characterized (Wardemann et al. Science 2003, 301(5638):1374-7), served as negative control.

2.2 Streptavidin ELISA

Specificity of antigen-binding was determined by binding to the unrelated protein Streptavidin by ELISA. The ELISA was performed as described in the O-antigen ELISA section, but instead of incubating the plate with biotinylated O-antigen dilutions 1×PBS was used.

2.3 Whole LPS Immunoblot:

2 ug LPS/sample of Klebsiella pneumoniae O-serotypes was diluted in SDS-containing loading dye (NEB) and heated for 5 min to 95° C. before applied to a gradient SDS-PAGE (anyKd Bio-Rad). LPS was transferred onto a nitrocellulose membrane and fixed by complete drying of the membrane. After re-activation of the membrane and an additional washing step, the membrane was placed overnight in a 4% BSA in TBS solution. Thus, the membrane was cut with a scalpel into appropriate pieces and incubated in a 2 ug/ml monoclonal human IgG1 antibody in TBS solution for 1.5 h at room-temperature. Subsequently, the membrane was washed 2-times with TBS for 5 min and incubated with anti-human IgG Fc HRP-coupled secondary antibody 1:10000 in TBS containing 1% BSA. After washing 3-times with TBS for 5 min, binding was detected using luminol-based detection (Pierce).

2.4 Ig Gene Analysis:

Human Ig genes were identified using the Ig gene reference database of IGMT Version 1.2.1 embedded into the NCBI Ig Blast using the IMGT or Kabat CDR definitions (M.-P. Lefranc et al., IMGT unique numbering for immunoglobulin and T cell receptor variable domains and Ig superfamily V-like domains. Dev. Comp. Immunol. 27, 55-77 (2003)). The best matched germline hit was identified. Somatic hypermutations (SHM) were counted from the end of the primer-binding region until the end of the IGHV, IGKV, or IGLV gene. Insertions or deletions regardless of their length were counted as one SHM.

2.5 Bioinformatics:

Plots were produced using Prism v6, Illustrator CS6 v16.0.3 (Adobe), Photoshop CS6 (Adobe) and R using the ggplot2 package. (H. Wickham, ggplot2: Elegant Graphics for Data Analysis (Springer-Verlag New York, 2009)).

Results

Fully Human Anti-Klebsiella pneumoniae O1-Antigen Antibodies

Biotinylated antigens in combination with fluorophore-coupled Streptavidin for their detection have been extensively applied to identify and isolate protein antigen-reactive B cells using flow cytometry (J. F. Scheid et al., A method for identification of HIV gp140 binding memory B cells in human blood. J. Immunol. Methods. 343, 65-7 (2009); 0. L. Rojas, C. F. Narváez, H. B. Greenberg, J. Angel, M. A. Franco, Characterization of rotavirus specific B cells and their relation with serological memory. Virology. 380, 234-42 (2008); P. F. Kerkman et al., Identification and characterisation of citrullinated antigen-specific B cells in peripheral blood of patients with rheumatoid arthritis. Ann. Rheum. Dis. 75, 1170-1176 (2016)).

Here, biotinylated K. pneumoniae O1 O-polysaccharide was used as bait and Streptavidin-conjugated fluorophores were used to identify and isolate polysaccharide antigen-reactive B cells (FIG. 3a). A rare population of O-antigen-binding memory B cells could be detected within the human peripheral blood memory B cell pool by flow cytometry (FIG. 3b). Single O1-antigen-binding B cells were isolated by fluorescence-activated cell sorting, their respective Ig heavy and associated Ig light chain genes were amplified by RT-PCR and Ig gene information was obtained by Sanger sequencing. The data show that the majority of O1-antigen-reactive B cells in peripheral blood express antibodies with various degrees of IGHV gene somatic hypermutations (FIG. 3c). Two monoclonal antibodies were cloned and recombinantly expressed from an IgM and IgA B cell, respectively, (KcPBO1-196 (herein also referred to as MPG-196) and KePBO1-084) as IgG1. Both antibodies were encoded by IGHV3 family genes and showed levels of somatic hypermutations above average. Both antibodies showed concentration-dependent binding to the biotinylated O-antigen bait in ELISA and whole O1 LPS in Immunolot (FIG. 3d and FIG. 3e) but lacked detectable levels of reactivity with Streptavidin by ELISA (FIG. 3f). Thus it was conclude that both antibodies are K. pneumoniae O1 O-antigen specific. Ig gene features are provided in the table shown in FIG. 3g.

### Example 2: Epitope of K. pneumoniae O1 Human mAb

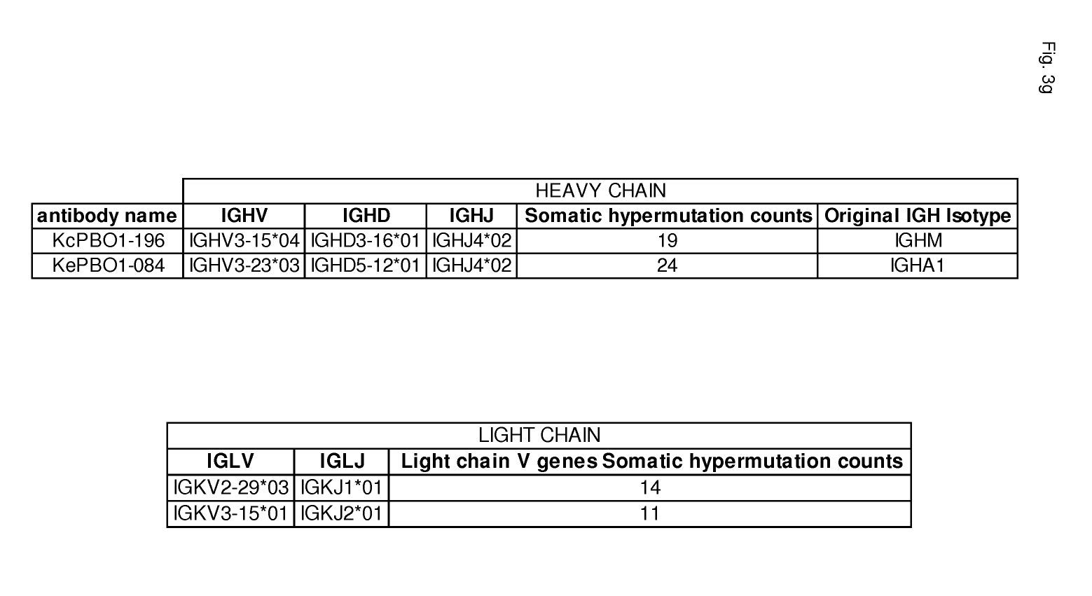

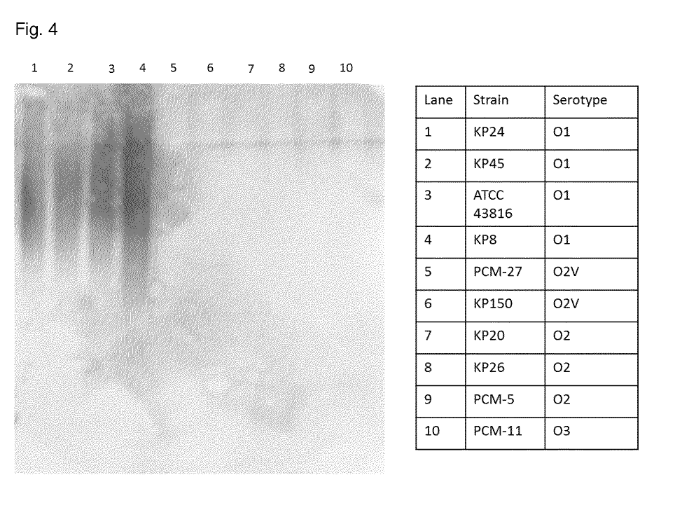

The O1 serotype LPS molecules have two different O-antigens (i.e. D-galactan-I located proximal to the cell anchored Lipid A-core and D-galactan-II capping D-gal-I) of which only D-gal-II is specific for O1. In order to determine whether the O1-specific human mAbs described above bind to the serotype determining D-gal-II epitopes, we tested reactivity of the mAbs to different Klebsiella serotypes. Binding specificity of mAb MPG-196 was confirmed with immunoblots using separated (SDS-PAGE) extracted purified LPS molecules blotted onto PVDF membranes (FIG. 4). The mAb was reacted for 1 h at 1 μg/ml concentration. Blot was developed using HRP-labelled anti-human IgG secondary antibody and luminography. The O1 specific mAb stained exclusively the long molecular weight LPS fraction of LPS type O1, suggesting a specific binding to galactan-II epitopes. The lack of binding to O2 type LPS molecules confirmed this specificity (since galactan-I or galactan-III, the other O-antigen repeating unit of serotype O1, is shared by O1 and O2 (galactan-I) or O2V (galactan-III) type LPS molecules).

### Example 3: O1 mAbs are Able to Bind to the Bacterial Surface Irrespective of the Capsular Type

It has been determined that mAbs bind to the surface and trigger Fc-dependent effector functions. K. pneumoniae shields its surface molecules by abundant capsular polysaccharide (CPS) that shows high structural variability. Still, it was shown that the discovered O1-specific mAb can efficiently bind to the bacterial surface in the presence of different CPS coats.

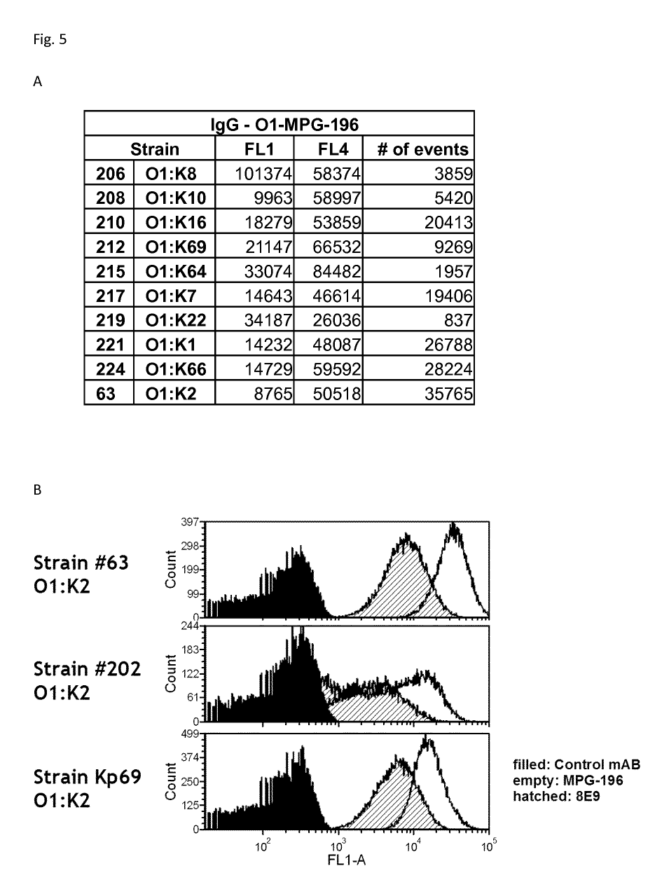

Mid-log cultures of K. pneumoniae O1 strains expressing different capsular types were stained with 40 μg/ml of mAb MPG-196 and subsequently with a secondary anti-human IgG labelled with Alexa 488. Additional incubation with 5 nM Syto62 dye (DNA-stain) enabled identification of the bacterial population. Fluorescence intensity was measured by flow cytometry. As summarized in FIG. 5A all 01 strains were strongly stained with MPG-196 irrespective of the capsular type (K-antigen). Furthermore, binding of MPG-196 was compared with that of another O1-specific mAb (a murine-human chimeric comparator mAb of lower affinity termed 8E9) on 3 different O1:K2 strains. K2 capsule is considered to be the thickest capsule type (also confirmed by staining —FIG. 5A) that is often associated with clinical isolates. Although both mAbs could bind to the surface of all O1:K2 strains, the staining with MPG-196 was higher in case of all strains (FIG. 5B).

### Example 4: Protective Capacity in a Murine Model of Bacteremia

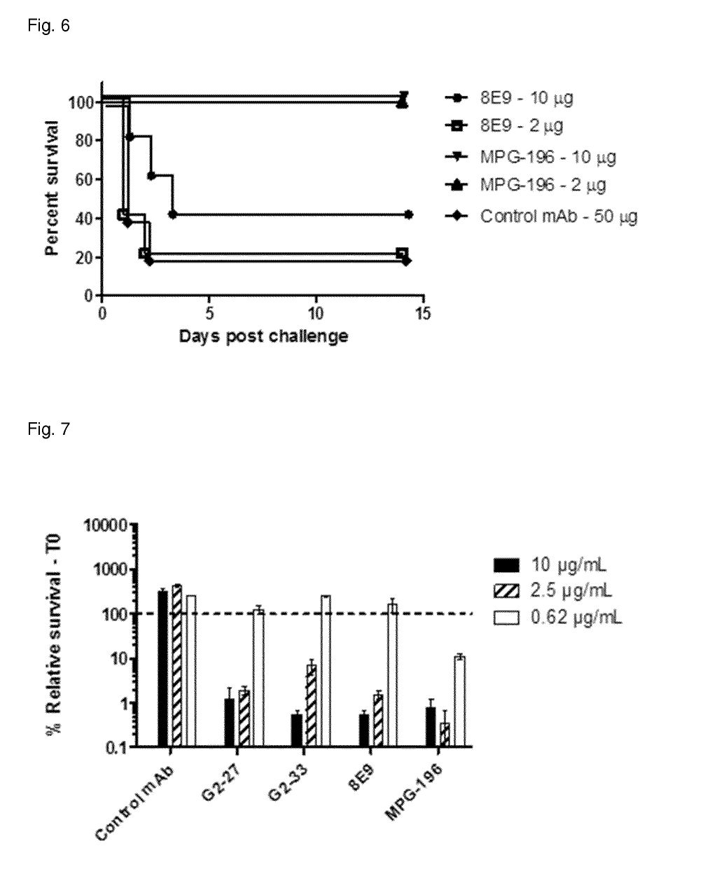

Protective efficacy of purified human (MPG-196) or murine-human chimeric (8E9) mAbs was tested in a murine model of K. pneumoniae bacteremia (FIG. 6). Groups of 5 mice were immunized intraperitoneally with either 2 or 10 μg of mAbs MPG-196 and 8E9. The control group received 50 μg isotype matched mAb with irrelevant specificity. 24 h later animals were challenged with a lethal dose (5×10⁶ CFU) of K. pneumoniae O1:K2 strain ATCC 43816. Lethality was monitored daily for 14 days. While MPG-196 showed full protection at both doses, the comparator O1 specific mAb (8E9) showed insignificant (at 10 μg/mouse) or no (at 2 μg/mouse) protection in this model. Similarly, O1-specific mAbs reported earlier (2) could elicit only partial protection at doses as high as 800 μg/mouse doses (and insignificant protection at lower doses) in a similar lethal murine model. Therefore, the protective efficacy of MPG-196 appears to be outstanding among the O1-specific mAbs reported to date.

### Example 5: Phagocyte-Independent Bactericidal Activity

Given that K. pneumoniae strains tend to infect immunocompromised patients with limited phagocytic capacity, it was considered advantageous to identify mAbs with direct bactericidal activity. Phagocyte-independent complement mediated bactericidal activity of the mAbs was tested in a serum bactericidal assay (SBA, FIG. 7). O1-specific humanized mAbs G2-27 and G2-33 (humanized medium affinity versions of 8E9) as well as their parental murine-human chimeric mAb (8E9) elicited dose-dependent complement-mediated bactericidal activity. Human mAb MPG-196 induced superior bactericidal activity at lower doses tested. No bactericidal activity was observed when using an isotype matched mAb with irrelevant specificity or upon heat-inactivation (56° C. for 30 min) of the sera used (not shown) corroborating an antibody dependent complement mediated killing.

### Example 6: Endotoxin Neutralization

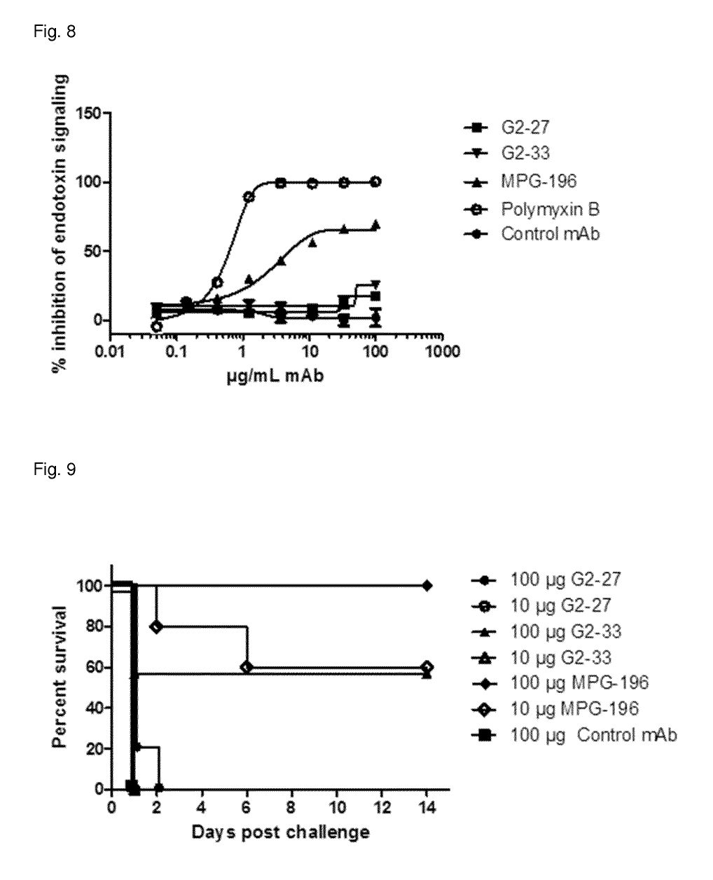

Neutralization of the endotoxic activity of extracted 01 LPS molecules was performed in a commercial cell line (HEK Blue, Invivogen) reporting on TLR-4 signaling (FIG. 8). Polymyxin B, a small antibiotic with proven Lipid A-binding potency was used as positive control, while an irrelevant isotype matched mAb served as negative control. MPG-196, unlike benchmark mAbs G2-27 and G2-33, showed a dose-dependent neutralization of endotoxin signalling. The inhibition plateaued between 60-70%, which originated from the specificity to galactan-II (i.e. LPS molecules lacking the outer galactan-II antigen were not neutralized).

In order to assess whether the in vitro observed partial neutralization of endotoxic activity has any in vivo relevance, the same mAbs were tested for protective efficacy in a mouse model of endotoxemia (FIG. 9). Groups of mice were passively immunized with 10 or 100 μg of the different mAbs. Twenty-four hours later mice were sensitized for endotoxin by GaIN (6), and simultaneously challenged with a lethal dose (2×10³ CFU) of strain #202. Survival was monitored for 14 days. mAb MPG-196 showed partial or full protection at the low and high doses, respectively. mAb G2-33 showed only partial protection at the higher dose, while G2-27 could not elicit any protection at any dose.

### Example 7: Binding Characteristics of O1 Specific mAbs

Binding to the biotinylated O1 antigen was measured by biolayer interferometry (BLI) using a fortéBIO Octet Red instrument (Pall Life Sciences). Biotinylated O1-polysaccharide as depicted schematically in FIG. 3a (5 μg/ml) was immobilized on the streptavidin (SA) sensor (fortéBIO, Pall Life Sciences) to give a sensor loadings of 0.5 nm. The association of the antibody (66.7 nM), was monitored for 600 s in solution (PBS, pH 7.2, plus 1% BSA), at 30° C. The sensors were then immersed in the same buffer, for 600 s, to monitor the dissociation of the antibody. The kinetic rate constants (kₒₙ and k_{dis}) were determined by fitting simultaneously the association and dissociation phases of each progress curve to a 1 to 1 binding model, using either a full or a partial fit (fortéBIO Analysis Software version 9.0). The dissociation constant (K_{d} value) was calculated as k_{dis}/kₒₙ.

Avid binding affinity of the mAbs to the O1 polysaccharide antigen was measured by biolayer interferometry in an avid state set-up, where biotinylated O1 antigen was immobilized on the sensor and the bivalent antibody was in solution. The fit of the data to a 1 to 1 binding model gives apparent dissociation constants for the avid interaction in the low nM range (˜4 nM) for MPG-196 and high nM range (300-700) for G2-27 and G2-33. The ˜2 log higher affinity of MPG-196 is essentially due to a lower k_{diss} (see table below).

Table: Binding (BLI) parameters of anti-Klebsiella 01 mAbs to purified biotinylated O1 antigen, at an antigen loading of 0.5 nm and a mAb concentration of 67 nM, in PBS, pH 7.2 plus 1% BSA, at 30° C. Association (kₒₙ) and dissociation (k_{off}) rate constants were determined by fitting the data to a 1 to 1 binding model, using a partial fit, except for MPG-196 (full fit). Where multiple measurements were done, values are given as average ±standard deviation.

| mAb | Response (nm) | K_{d} (M) | kₒₙ (1/Ms) | k_{dis} (1/s) |
|---|---|---|---|---|
| 8E9 | 0.87 ± 0.03 | (2.14 ± 0.12)E−07 | (2.29 ± 0.19)E05 | (4.90 ± 0.25)E−02 |
| MPG-196 | 6.36 | 3.37E−09 | 1.22E05 | 4.09E−04 |
| G2-27 | 0.84 ± 0.23 | (2.90 ± 1.20)E−07 | (1.23 ± 0.22)E05 | (3.63 ± 1.54)E−02 |
| G2-33 | 0.62 ± 0.16 | (7.36 ± 2.46)E−07 | (8.48 ± 6.76)E04 | (5.53 ± 3.66)E−02 |

### REFERENCES

1. Held T K, Jendrike N R, Rukavina T, Podschun R, Trautmann M. 2000. Binding to and opsonophagocytic activity of O-antigen-specific monoclonal antibodies against encapsulated and nonencapsulated Klebsiella pneumoniae serotype O1 strains. Infect. Immun. 68:2402-2409.
2. Rukavina T, Ticac B, Susa M, Jendrike N, Jonjic S, Lucin P, Marre R, Doric M, Trautmann M. 1997. Protective effect of antilipopolysaccharide monoclonal antibody in experimental Klebsiella infection. Infect. Immun. 65:1754-1760.
3. Trautmann M, Ruhnke M, Rukavina T, Held T K, Cross A S, Marre R, Whitfield C. 1997. O-antigen seroepidemiology of Klebsiella clinical isolates and implications for immunoprophylaxis of Klebsiella infections. Clin. Diagn. Lab Immunol. 4:550-555.
4. Whitfield C, Richards J C, Perry M B, Clarke B R, MacLean L L. 1991. Expression of two structurally distinct D-galactan O antigens in the lipopolysaccharide of Klebsiella pneumoniae serotype O1. J. Bacteriol. 173:1420-1431.
5. Kol O, Wieruszeski J M, Strecker G, Fournet B, Zalisz R, Smets P. 1992. Structure of the O-specific polysaccharide chain of Klebsiella pneumoniae O1 K2 (NCTC 5055) lipopolysaccharide. A complementary elucidation. Carbohydr. Res. 236:339-344.
6. Galanos C, Freudenberg M A, Reutter W. 1979. Galactosamine-induced sensitization to the lethal effects of endotoxin. Proc. Natl. Acad. Sci. U.S.A 76:5939-5943.

1-15. (canceled)

16. A monoclonal antibody that specifically recognizes Klebsiella pneumoniae serotype O1, which is capable of neutralizing the LPS endotoxin activity, which antibody is selected from any of
a) an antibody comprising the CDR1-CDR6 sequences of any one of the antibodies listed in Table 1a or Table 1b; or
b) an antibody comprising the the VH and VL sequences of any one of the antibodies depicted in FIG. 2b; or
c) an antibody which is a functionally active variant of a parent antibody that is any one of the antibodies of a) or b), which functionally active variant specifically recognizes Klebsiella pneumoniae serotype O1 and is capable of neutralizing the LPS endotoxin activity, and comprises at least one point mutation in any of the CDR, wherein the number of point mutations is either 0, 1, 2, or 3 point mutations in each of the CDR sequences, wherein the sequence identity in each of the CDR sequences is at least 60% compared to the respective CDR sequences of the parent antibody.

17. The antibody of claim 16, which is
A)
selected from the group consisting of group members i) to ii), wherein
i)
is an antibody which comprises
a) CDR1 consisting of the amino acid sequence SEQ ID 9;
b) CDR2 consisting of the amino acid sequence SEQ ID 10;
c) CDR3 consisting of the amino acid sequence of SEQ ID 11;
d) CDR4 consisting of the amino acid sequence SEQ ID 15;
e) CDR5 consisting of the amino acid sequence SEQ ID 16; and
f) CDR6 consisting of the amino acid sequence of SEQ ID 17;
ii)
is an antibody which comprises
a) CDR1 consisting of the amino acid sequence SEQ ID 12;
b) CDR2 consisting of the amino acid sequence SEQ ID 13;
c) CDR3 consisting of the amino acid sequence of SEQ ID 14;
d) CDR4 consisting of the amino acid sequence SEQ ID 18;
e) CDR5 consisting of the amino acid sequence SEQ ID 19; and
f) CDR6 consisting of the amino acid sequence of SEQ ID 20;
wherein CDR sequences are designated according to the numbering system of Kabat;
or
B) an antibody which is the functionally active variant of a parent antibody that is any of the group members of A.

18. The antibody of claim 16, which is
A)
selected from the group consisting of group members i) to ii), wherein
i)
is an antibody which comprises
a) CDR1 consisting of the amino acid sequence SEQ ID 21;
b) CDR2 consisting of the amino acid sequence SEQ ID 22;
c) CDR3 consisting of the amino acid sequence of SEQ ID 23;
d) CDR4 consisting of the amino acid sequence SEQ ID 27;
e) CDR5 consisting of the amino acid sequence SEQ ID 28; and
f) CDR6 consisting of the amino acid sequence of SEQ ID 29;
and
ii)
is an antibody which comprises
a) CDR1 consisting of the amino acid sequence SEQ ID 24;
b) CDR2 consisting of the amino acid sequence SEQ ID 25;
c) CDR3 consisting of the amino acid sequence of SEQ ID 26;
d) CDR4 consisting of the amino acid sequence SEQ ID 30;
e) CDR5 consisting of the amino acid sequence SEQ ID 31; and
f) CDR6 consisting of the amino acid sequence of SEQ ID 32;
wherein CDR sequences are designated according to the numbering system of IMGT;
or
B) an antibody which is the functionally active variant of a parent antibody that is any of the group members of A.

19. The antibody of claim 16, which is
A)
selected from the group consisting of group members i) to ii), wherein
i)
is an antibody which comprises
a) VH consisting of the amino acid sequence SEQ ID 5; and
b) VL consisting of the amino acid sequence SEQ ID 7;
and
ii)
is an antibody which comprises
a) VH consisting of the amino acid sequence SEQ ID 6; and
b) VL consisting of the amino acid sequence SEQ ID 8;
or
B) an antibody which is the functionally active variant of a parent antibody that is any of the group members of A.

20. The antibody of claim 16, which is a high affinity antibody binding the epitope with a K_{D} of less than 10⁻⁸M as determined by biolayer interferometry for bivalent binding.

21. The antibody of claim 16, which is neutralizing endotoxin of Klebsiella pneumoniae strains expressing the the D-galactan-II epitope.

22. The antibody of claim 16, which is any one of a full-length monoclonal antibody, an antibody fragment thereof, or a fusion protein, each comprising at least VH and VL antibody domains incorporating a binding site recognizing the D-galactan-II epitope.

23. The antibody of claim 16, which is of human origin, or an affinity matured variant thereof, specifically wherein the antibody is a non-naturally occurring antibody which comprises an artificial amino acid sequence, specifically wherein the antibody is any one of an IgA, IgM, or an IgG isotype switch variant thereof.

24. The antibody of claim 16, for use in treating a subject at risk of or suffering from K pneumoniae infection or colonization to limit the infection in the subject or to ameliorate a disease condition resulting from said infection, preferably for treatment or prophylaxis of any of primary and secondary bacteremia, pneumonia, urinary tract infection, liver abscess, peritonitis, or meningitis.

25. The antibody for use according to claim 24, wherein the subject is suffering from endotoxemia caused by K pneumoniae.

26. A pharmaceutical preparation comprising the antibody of claim 16, and a pharmaceutically acceptable carrier or excipient in a parenteral formulation.

27. An isolated nucleic acid encoding the antibody of claim 16.

28. An expression cassette or a plasmid comprising a coding sequence to express a proteinaceous construct comprising a VH and/or VL of the antibody of claim 16.

29. A host cell comprising the expression cassette or a plasmid of claim 28.

30. A method of producing the antibody of claim 16, wherein the host cell of claim 14 is cultivated or maintained under conditions to produce said antibody.


SEQUENCE LISTING (32 sequences)

SEQ ID NO: 1 - DNA, 352 bp, Artificial Sequence
  VH
gaagtgcagctggtggagtctgggggagacttggtaaaacctggggggtcccttagactctcctgtgcagtctctggactcactttcagtgacgcctggctgaactgggtccgccagtctccagggaaggggccggagtgggttggccgtattgagagcaaagcttatggtgagacaacacactactctgcacccgtgagaggcagattcaccatctcaagagatgattcaaaaaacacgatgtatctgcaaatgaacagcctgaaaaccgaagacacagccgtctattactgtgccacagatgcacagtgggggaggtggggccagggaaccctggtcaccgtctcctcag

SEQ ID NO: 2 - DNA, 364 bp, Artificial Sequence
  VH
gaggtgcagctgttggagtctgggggaggccttgtacagccaggggggtccctgagactctcctgtgcagcctctggattcacctttagcaactttgccatgagctgggtccgccaggctccagggaaggggctggaatgggtctcatctatttttatcggtgctgctaacccaagccatgcagactctgtgaggggccggttcaccatctccagagatgattccaagaacacactgtatctgcaaatgaacagcctgagggccgaagacacggccgtatattactgtgcgaaagatgagggtggctccaaccttgactaccttgacaactggggccagggaaccctggtcaccgtctcctcag

SEQ ID NO: 3 - DNA, 328 bp, Artificial Sequence
  VL
gatattgtgatgacccagactccactctctctgtccgtcacccctggacagccggcctccatctcttgcaagtctagtcagagcctcctaaatagcgatggaaagaccaacttgtattggtacctgcagaagccaggccagcctccacaactcctgatgtatgaggtttccagccggttttctggagtgcccgatagattcagtggcagcgggtcagggacagatttcacactgaaaatcagccgggtggaggctgaggatgttgggatttattactgcatgcaggatccgtggacgttcggccaagggaccaaggtggaaatcaaac

SEQ ID NO: 4 - DNA, 325 bp, Artificial Sequence
  VL
gaaatagtgatgacgcagtctccagccaccctgtctgtgtctccaggggaaagagccaccctcttctgtagggccagtcagtatattggctacaacttagcctggtaccagcagaaacctggccaggctcccaggctcctcatctatggtgcatccaccagggccactggtatcccagccaggttcagtggcagtgggtctgggacagacttcactctcaccatcagcagcctgcagtctgaagactttgcactttattactgtcagcagtatcataactggcctccgtacacctttggccaggggaccaagctggagatcaaac

SEQ ID NO: 5 - protein, 117 aa, Artificial Sequence
  VH
EVQLVESGGDLVKPGGSLRLSCAVSGLTFSDAWLNWVRQSPGKGPEWVGRIESKAYGETTHYSAPVRGRFTISRDDSKNTMYLQMNSLKTEDTAVYYCATDAQWGRWGQGTLVTVSS

SEQ ID NO: 6 - protein, 121 aa, Artificial Sequence
  VH
EVQLLESGGGLVQPGGSLRLSCAASGFTFSNFAMSWVRQAPGKGLEWVSSIFIGAANPSHADSVRGRFTISRDDSKNTLYLQMNSLRAEDTAVYYCAKDEGGSNLDYLDNWGQGTLVTVSS

SEQ ID NO: 7 - protein, 109 aa, Artificial Sequence
  VL
DIVMTQTPLSLSVTPGQPASISCKSSQSLLNSDGKTNLYWYLQKPGQPPQLLMYEVSSRFSGVPDRFSGSGSGTDFTLKISRVEAEDVGIYYCMQDPWTFGQGTKVEIK

SEQ ID NO: 8 - protein, 108 aa, Artificial Sequence
  VL
EIVMTQSPATLSVSPGERATLFCRASQYIGYNLAWYQQKPGQAPRLLIYGASTRATGIPARFSGSGSGTDFTLTISSLQSEDFALYYCQQYHNWPPYTFGQGTKLEIK

SEQ ID NO: 9 - protein, 5 aa, Artificial Sequence
  CDR
DAWLN

SEQ ID NO: 10 - protein, 19 aa, Artificial Sequence
  CDR
RIESKAYGETTHYSAPVRG

SEQ ID NO: 11 - protein, 6 aa, Artificial Sequence
  CDR
DAQWGR

SEQ ID NO: 12 - protein, 5 aa, Artificial Sequence
  CDR
NFAMS

SEQ ID NO: 13 - protein, 17 aa, Artificial Sequence
  CDR
SIFIGAANPSHADSVRG

SEQ ID NO: 14 - protein, 12 aa, Artificial Sequence
  CDR
DEGGSNLDYLDN

SEQ ID NO: 15 - protein, 16 aa, Artificial Sequence
  CDR
KSSQSLLNSDGKTNLY

SEQ ID NO: 16 - protein, 7 aa, Artificial Sequence
  CDR
EVSSRFS

SEQ ID NO: 17 - protein, 6 aa, Artificial Sequence
  CDR
MQDPWT

SEQ ID NO: 18 - protein, 11 aa, Artificial Sequence
  CDR
RASQYIGYNLA

SEQ ID NO: 19 - protein, 7 aa, Artificial Sequence
  CDR
GASTRAT

SEQ ID NO: 20 - protein, 10 aa, Artificial Sequence
  CDR
QQYHNWPPYT

SEQ ID NO: 21 - protein, 8 aa, Artificial Sequence
  CDR
GLTFSDAW

SEQ ID NO: 22 - protein, 10 aa, Artificial Sequence
  CDR
IESKAYGETT

SEQ ID NO: 23 - protein, 8 aa, Artificial Sequence
  CDR
ATDAQWGR

SEQ ID NO: 24 - protein, 8 aa, Artificial Sequence
  CDR
GFTFSNFA

SEQ ID NO: 25 - protein, 8 aa, Artificial Sequence
  CDR
IFIGAANP

SEQ ID NO: 26 - protein, 14 aa, Artificial Sequence
  CDR
AKDEGGSNLDYLDN

SEQ ID NO: 27 - protein, 11 aa, Artificial Sequence
  CDR
QSLLNSDGKTN

SEQ ID NO: 28 - protein, 3 aa, Artificial Sequence
  CDR
EVS

SEQ ID NO: 29 - protein, 6 aa, Artificial Sequence
  CDR
MQDPWT

SEQ ID NO: 30 - protein, 6 aa, Artificial Sequence
  CDR
QYIGYN

SEQ ID NO: 31 - protein, 3 aa, Artificial Sequence
  CDR
GAS

SEQ ID NO: 32 - protein, 10 aa, Artificial Sequence
  CDR
QQYHNWPPYT